(19) United States
(12) Patent Application Publication
(10) Pub. No.: US 2006/0029937 A1
(43) Pub. Date: Feb. 9, 2006

(54) Analysis of mixtures of nucleic acid fragments

(75) Inventor: Achim Fischer, Heidelberg (DE)

Correspondence Address: FOLEY AND LARDNER LLP;SUITE 500, 3000 K STREET NW, WASHINGTON, DC, 20007, US

(73) Assignee: AXARON BIOSCIENCE AG, 69120 HEIDELBERG (DE)

(21) Appl. No.: 10/504,847

(22) PCT Filed: Feb. 27, 2003
(86) PCT No.: PCT/EP03/02032

§ 371 Date: Oct. 1, 2004

(30) Foreign Application Priority Data
Feb. 27, 2002 (DE) 102 08 333.9

(51) Int. Cl. C12Q 1/68; C12P 19/34

(52) U.S. Cl. 435/6; 435/91.2

(57) ABSTRACT

The invention relates to a method of analyzing nucleic acid fragments, comprising the following steps: a) providing at least one mixture of nucleic acid fragments which have at least one recognition site for a restriction endonuclease cutting outside its recognition site, b) incubating at least a fraction of said mixture of nucleic acid fragments of step (a) with at least one restriction endonuclease whose cleavage site is located outside its recognition site, c) identifying one or more nucleotides of the cut nucleic acid fragments of (b) and, where appropriate, identifying further fragment-specific properties of said cut nucleic acid fragments of (b), said identification(s) being carried out simultaneously for a plurality of or for all nucleic acid fragments.

The invention relates to a method of analyzing nucleic acid fragment mixtures and to applying said mixture to gene expression analysis.

Methods of sequencing nucleic acid mixtures as can be obtained, for example, by “reverse transcribing” mRNA molecules to cDNA molecules have been disclosed in the prior art. The cDNA molecules obtained by reverse transcribing numerous different mRNA-molecules isolated from a cell or a tissue are cloned, usually into plasmid or phage vectors, and then sequenced “clone by clone” (Sambrook, Maniatis, Fritsch. Molecular cloning: a laboratory manual, Cold Spring Harbor/N.Y. 1989), said sequencing usually being carried out in a “strand-synthesizing” manner according to the chain termination principle of Sanger or in a “chain-degrading” manner in the sequencing according to Maxam and Gilbert. In each case, different molecules are thus separated by isolation in the form of plasmids transformed into bacterial cells, followed by multiplying the isolated molecules to give identical copies, thus obtaining “pure” signals (i.e. signals derived from identical molecules) in the sequencing process. Said procedure is suitable, for example for “EST sequencing” (EST=expressed sequence tag), which involves partially sequencing numerous clones obtained in the manner described and listing the sequence results obtained. Depending on whether or not the sequence library has previously been normalized, the relative frequency with which a particular cDNA or a particular EST has been sequenced reflects the abundance of the corresponding transcript. Thus, EST sequencing may be used not only for detecting expressed genes but also for comparing strengths of expression between various biological samples (cf. for example, Lee et al., Proc. Natl. Acad. Sci. U.S.A. 92 (1995), 8303-8307). However, the method of EST-sequencing for, where appropriate comparative, expression profiling is very laborious, especially due to said connection between the relative abundance of the transcripts and the relative abundance of the clones, since some transcripts (for example so-called housekeeping-genes) are much more abundant than other transcripts and thus clones of such abundant transcripts may need to be sequenced several hundred to several thousand times in order to be able to record, on the other hand, also less abundant transcripts.

In the past, a plurality of alternative methods have been described in which merely fragments of rather than complete cDNA molecules are analyzed. Particular mention must be made of the methods of RAP (RNA arbitrarily primed PCR; Welsh et al., Nucleic Acids Res. 20: 4965-70) and Differential Display (Liang and Pardee, Science 257: 967-971), in which transcript fragments are amplified by means of PCR using short primers of a randomly selected sequence. These fragments whose length again can greatly vary from transcript to transcript are fractionated according to their size by means of gel electrophoresis and detected. In this case, at least theoretically, the abundance of a transcript is no longer represented by the frequency of an event, for example the frequency with which a clone representing said transcript appears, but by the intensity of the particular band. This substantially eliminates the redundance which distinguishes EST sequencing of the prior art, thus reducing costs. In order to enable individual fragments to be sequenced, the particular bands are isolated from the gel, reamplified by means of PCR and cloned. More modern variants of this method, as described, for example, in EP 0 743 367, are based on generating fragments by means of restriction digestion of double-stranded cDNA, thereby distinctly increasing the reproducibility of the fragment patterns obtained. Nevertheless, methods of this kind still have the disadvantage of products contaminated by other undesired DNA fragments frequently being obtained when isolating bands from a gel. Furthermore, the isolation and cloning of individual bands requires a lot of work so that identification of fragments without prior isolation would be very desirable. Sutcliffe et al. (Proc. Natl. Acad. Sci. U.S.A. 97: 1976-1981) describe a method, named “TOGA”, of converting mRNA-molecules to cDNA restriction fragments which are fractionated by means of capillary gel electrophoresis. A signature (i.e. a collection of fragment-specific information such as, for example, fragment length, partial nucleotide sequence, information about position and/or orientation of the fragment within the starting cDNA etc.) is defined for fragments of interest (which indicate differentially expressed genes by differences in the intensities of the bands in question when comparing different preparations) and in this case consists of an 8 bp partial sequence which is known for each fragment and information about the distance of this sequence from the 3′ end of the fragment. By means of this signature it is possible to identify genes having the same signature by screening sequence databases. If the signature generated is error-free, cDNA fragments may be assigned to the corresponding genes without having to isolate and sequence said fragments. The method described, however, has disadvantages which result in said signatures being unreliable: (1) the identification of 4 nucleotides of the 8 bp sequence, which is carried out by “invasive” or “selective” amplification primers, is inaccurate, since often primers are also incorporated whose selective portion, namely the nucleotides located at the 3′ end, are not perfectly complementary to the template, and (2) determining the fragment length via electrophoretic mobility is inaccurate, since the mobility of a fragment depends, besides on the length, also additionally on the G/C content and on the exact sequence of said fragment (cf. Forensic Sci. Int. 94, 155-6 [1998]; regarding the term complementarity, cf. the base pair rules known from the literature, for example in Ausubel et al., Current Protocols in Molecular Biology (1999), John Wiley & Sons). Therefore, a wrong length is often assumed. However, a wrong length and/or a wrong sequence result in a signature determined for a given fragment not indicating the gene to be identified but rather the corresponding database search producing either no result or a wrong result. Similar restrictions apply to a comparable method, named “GeneCalling”, in which cDNA is subjected to double digestions with various combinations of restriction endonucleases (Shimkets et al., Nature Biotechnol. 17, 798-803 [1999]). The fragments obtained are fractionated by gel electrophoresis, their length and, from that, the distance between the two restriction endonuclease recognition sites on which the formation of a fragment is based are determined, and signatures are generated which consist of the sequence of the first recognition site, the sequence of the second recognition site and the assumed distance of the two recognition sites from one another (expressed in base pairs). By means of these signatures, database searches are carried out in order to assign detected fragments to those genes from which said fragments derive. Here too, it is evident that a high proportion of wrong assignments of database entries to detected fragments occurs, owing to great uncertainties in the determination of fragment sizes on the basis of fragment mobilities.

It was therefore the object of the present invention to assign to nucleic acid fragments present in a mixture signatures which do not have the disadvantages of the prior art.

The object of the invention is achieved by a method of analyzing nucleic acid fragments, comprising the steps:
(a) providing a mixture of nucleic acid fragments which have at least one recognition site for a restriction endonuclease cutting outside its recognition site,
(b) incubating at least a fraction of said mixture of nucleic acid fragments of step (a) with at least one restriction endonuclease whose cleavage site is located outside its recognition site,
(c) identifying in each case one or more nucleotides of the cut nucleic acid fragments of (b), said identification being carried out simultaneously for a plurality of or all nucleic acid fragments.

The object of the invention is furthermore achieved by a method of analyzing nucleic acid fragments, comprising the steps:
(a) providing a mixture of nucleic acid fragments which have at least one recognition site for a restriction endonuclease cutting outside its recognition site,
(b) incubating at least a fraction of the mixture of nucleic acid fragments of step (a) with at least one restriction endonuclease whose cleavage site is located outside its recognition site and which generates protruding ends of known position and length, but unknown sequence,
(c) identifying in each case one or more nucleotides of said protruding ends of the cut nucleic acid fragments of (b), said identification being carried out simultaneously for a plurality of or all nucleic acid fragments.

The mixture of nucleic acid fragments preferably is, where appropriate amplified, restriction fragments of cDNA or of genomic DNA. The fragments or part of said fragments may be flanked by sequence regions common to all or to some fragments. Said common sequence regions may be, for example, linkers or adapters added to the fragments, i.e. double-stranded nucleic acid fragments which are available, for example, by hybridizing two oligonucleotides essentially or at least partially complementary to one another. Adapters are typically characterized by a length of between 5 and 200 nucleotides, preferably between 10 and 80 nucleotides, particularly preferably between 15 and 40 nucleotides. Preferably, the fragments exhibit a characteristic size distribution with a smallest occurring size, a largest occurring size and an average size, with said size being influenced or determined by the positions and/or the frequency of the recognition site or recognition sites for the restriction endonuclease or restriction endonucleases used for generating said fragments, it being necessary here, of course, to take into account also the length of linkers or adapters which may have been added. In a preferred embodiment, a mixture of nucleic acid fragments, preferably double-stranded cDNA, is cut with at least one restriction endonuclease which preferably has a four-base recognition sequence. Examples of suitable restriction endonucleases are AluI, BfaI, BstUI, ChaI, Csp6I, CviJI, CviJI, DpnI, DpnII, HaeIII, HhaI, HinP1I, HpaII, HpyCH4 IV, HpyCH4 V, MboI, MseI, MspI, NlaIII, RsaI, Sau3aI, TaiI, TaqI, Tsp509I. Frequently, linker molecules are attached, usually via enzymatic ligation, to in each case one or both ends of the fragments obtained in this way. This may be carried out without after-treatment of the fragments when fragment ends and linker ends are compatible with one another, i.e. are blunt or have protruding ends complementary to one another. However, it is also possible to subject the fragment ends to an after-treatment in order to achieve complementarity. For example, single-stranded fragment ends can be removed by means of a nuclease or else, in the case of 5′-protruding ends, filled in by means of a polymerase and thus converted to blunt ends if it is intended to attach linkers with blunt ends. Another example of an after-treatment of fragment ends is partial filling-in which may prevent two fragment ends from ligating to one another, which is usually undesired. For example, it is possible for a palindromic and thus self-complementary protruding end of the sequence 3′-CTAG-5′, which has been generated by treatment with the restriction endonuclease Sau3al, to be converted to a no longer self-complementary protruding end of the sequence 5′-TAG-3′ by treatment with a polymerase in the presence of dGTP. It would be possible to attach to such a protruding end then only linkers having a complementary protruding end, 5′-ATC-3′, thereto; a ligation of two fragment ends to one another would no longer be possible. In order to prepare a desired subgroup of fragments, attachment of the linkers is followed, where appropriate, by amplification with one or more PCR primers directed against the added linkers or with one or more PCR primers directed against the added linkers and additionally one PCR primer directed against a terminal region of the original nucleic acid fragments, preferably of the starting cDNA molecules. Suitable for this is, for example, the region which has been introduced by the cDNA primer used for cDNA synthesis or a region which has been added artificially to the 5′ end of the mRNA used for cDNA synthesis or to the 3′ end of the first-strand cDNA. In the first case, “cDNA-internal” fragments are amplified, i.e. fragments which, prior to attachment of the linkers, had ends generated on both sides by restriction cleavage, and in the second case “terminal” fragments are amplified which, prior to attachment of the linkers, had one end generated by restriction cleavage and whose other end is identical to the 3′ end or to the 5′ end of the original nucleic acid fragments or of the starting cDNA. The cDNA primer used in this embodiment is preferably an oligo-dT primer which may have at its 3′ end and/or at its 5′ end an extension by one or more nucleotides of which at least some are not “T”. If two or more restriction endonucleases generating different ends are used for fragment generation, it is possible to use in the subsequent step different linkers one part of which can be attached to one type of end and another part can be attached to a different kind of end. If these linkers differ from one another not only in their ends and thus in their compatibility (i.e. their attachability) to the fragment ends, but also in their remaining sequence, then it is possible to amplify, by appropriately choosing the primers in a subsequent PCR amplification, specifically particular fragments (those to whose linker sequences the chosen primers can bind under the amplification conditions set), while particular other fragments (those to whose linker sequences the chosen primers cannot bind) remain unamplified. It is also possible to amplify selectively particular fragments by using invasive primers which have been extended at their 3′ end by one or more additional selective bases, compared to the linker sequence common to all fragments (see, for example, EP 0 743 367). WO 94/01582 describes yet another possibility of selective isolation or amplification which may be applied in the course of the method of the invention.

Restriction endonucleases cutting outside their recognition site are those restriction endonucleases for which the partial sequence causing the enzyme activity (the recognition site), which is usually a region of double-stranded DNA consisting of 4-8 base pairs and at which the enzyme binds to the DNA double strand, and the cleavage site, i.e. the region of said DNA double strand, in which the sugar phosphate backbone of the DNA strands is hydrolytically cut, are offset with respect to one another on at least one of the two strands forming said double strand. Examples thereof are type IIs restriction endonucleases such as, for example, FokI [cutting characteristics GGATG(9/13): the “upper” strand is cut 9 bases away from the recognition site GGATG, the “lower” strand is cut 13 bases away from the recognition site] or BtsI [cutting characteristics GCAGTG(2/0)] or the restriction endonuclease BcgI [cutting characteristics (10/12)CGANNNNNNTGC (12/10): both strands are cut in each case once upstream of and once downstream of the recognition site]. Other examples are the restriction endonucleases AarI, AceIII, AloI, AlwI, BaeI, Bbr7I, BbsI, BbvI, BceAI, BcefI, BciVI, BfuAI, BmrI, BplI, BpmI, BpuEI, BsaI, BsaXI, BscAI, BseMII, BseRI, BsgI, BsmAI, BsmBI, BsmFI, Bsp24I, BspCN I, BspMI, BsrDI, BstF5I, CjeI, CjePI, EarI, EciI, Eco57I, Eco57MI, FalI, FauI, HaeIV, HgaI, Hin4I, HphI, MboII, MmeI, Mn/I, PleI, PpiI, PsrI, RleAI, SapI, SfaNI, Sth132I, StsI, TaqII, TspDT I, TspGW I, Tth111II. The method of the invention is carried out by giving preference to using those restriction endonucleases which generate single-stranded protruding ends which may be either 3′-protruding or 5′-protruding ends. If restriction endonucleases which generate blunt ends (e.g. MlyI, cutting characteristics GAGTC(5/5), or SspD5 I, GGTGA(8/8)) are intended to be used, said blunt ends may be converted in an additional step to protruding ends. This may be carried out, for example, by incubation with T4 DNA polymerase in the presence of a selected nucleotide triphosphate; the exonuclease activity of said T4 DNA polymerase then degrades one of the two strands in the 3′→5′ direction, until reaching the first “same-name” nucleotide in the strand (i.e. until the first “G” when the nucleotide triphosphate used was dGTP, for example; see Ausubel et al., Current Protocols in Molecular Biology (1999), John Wiley & Sons). Another type of restriction endonucleases cutting outside their recognition site are enzymes whose recognition site is interrupted by a sequence of random or substantially random nucleotides. Examples thereof are enzymes such as XcmI (cutting characteristics CCANNNNN/NNNNTGG) or SfiI (cutting characteristics GGCCNNNN/NGGCC). A special case which must also be taken into account of restriction endonucleases cutting outside their recognition site are “nicking endonucleases” which merely cut one strand of a nucleic acid double strand. Examples of those endonucleases are N.AlwI (GGATCNNN/N) and N.BstNBI (GAGTCNNN/N), which in each case cut only the sense strand at the position indicated by “l”. If it is intended to use such endonucleases for carrying out the method of the invention, then care must be taken of the fragments in question after cleavage to be converted to fragments which have a single-stranded protruding end. This may be carried out, for example, by one of the two following measures: (1) “melting off” a short single strand adjacent to the cleavage site by alkaline or beat denaturation, with the remaining fragment being intended to remain double-stranded, (2) incubation with a further restriction endonuclease which can also cut the counterstrand of the strand cut (or still to be cut) by means of said “nicking endonuclease”.

The recognition site for a restriction endonuclease cutting outside its recognition site, which recognition site appears in the nucleic acid fragments of the fragment mixture in (a), is preferably located within the terminal sequence regions common to many or all of the fragments of the mixture, thus, in particular, in the sequence regions of the adapters or linkers added to said fragments. In this case, the enzyme and the position of the recognition site must be chosen so as for the restriction endonuclease or restriction endonucleases to cause a “proximal” cut and for the particular nucleic acid fragment to be cut in the fragment-specific region which is located outside the flanking linker regions common to all or many fragments. In a particularly preferred embodiment, recognition sites of the restriction endonucleases to be used, which are, where appropriate, present in individual fragments and which are located outside the flanking linker regions common to all or many fragments, are protected from being recognized by the corresponding restriction endonuclease. Particular recognition sites for particular restriction endonucleases can be protected in this way according to the prior art, for example, by incorporating methylated nucleotides such as methyl-dCTP, for example. Alternatively, protection against restriction-endonucleolytic cleavage may also be obtained by using a methylase associated with the restriction endonuclease selected. For example, the enzyme BamHI methylase converts recognition sites of the restriction endonuclease BamHI to their C-methylated form which is no longer recognized and cut by BamHI. The enzyme CpG methylase methylates CG dinucleotides, thereby preventing, for example, a DNA fragment comprising the sequence CGTCTC from being cut by the restriction endonuclease BsmBI (cutting characteristics CGTCTC(1/5)). In any case, the above measures ensure that each nucleic acid fragment present in the mixture is cut only at exactly one predetermined position in the course of a restriction digestion. It would furthermore be possible to incubate the starting nucleic acid molecules (preferably cDNA or genomic DNA) used for generating the nucleic acid fragments of (a) with the restriction endonuclease of step (b) beforehand, then to treat them, as described above, with at least one further restriction endonuclease which usually cuts frequently, to attach to the ends generated by the latter linker molecules and to carry out a PCR amplification using primers directed against the terminal linker molecules. This procedure ensures that the nucleic acid fragments in step (b) are cut only at the desired sites determined by the added linkers, since fragments having their “own”, fragment-internal recognition site for said restriction endonuclease can no longer be amplified after cleavage and thus do not appear in the fragment mixture according to (a).

Identification of in each case one or more nucleotides of the cut nucleic acid fragments may be carried out in several different ways. Particularly suitable here are three preferred procedures which, however, should not preclude other procedures:
1. Extension of recessed 3′ ends by dideoxynucleotide triphosphates (“ddNTPs”) used for the nowadays common sequencing according to Sanger or else by acyclic nucleotides (i.e. by so-called “termination nucleotides” or “chain terminators”), with each strand to be filled in being extended by exactly one nucleotide and chain extension terminating thereafter, since a 3′-OH group is no longer available. Since the incorporation is sequence-specific, the nucleotide opposite the nucleotide incorporated in the double strand is unambiguously identifiable. The termination nucleotides preferably carry labeling groups, on the basis of which incorporation can be detected. In a particularly preferred embodiment, the four dideoxynucleotides carry four different labeling groups, in particular four different fluorophores. It is then possible, on the basis of the fluorescence activity, to detect which of the four termination nucleotides has been incorporated and, accordingly, also which nucleotide is present on the particular counterstrand. Carrying out this first embodiment requires of course that the nucleic acid fragments of (c) have recessed 3′ ends which therefore can be filled in by means of a polymerase. This may readily be ensured by an appropriate choice of the restriction endonuclease of (b). Suitable are in particular the following type IIs restriction endonucleases: AarI, AceIII, AlwI, Bbr7I, BbsI, BbvI, BceAI, BcefI, BfuAI, BsaI, BscAI, BsmAI, BsmBI, BsmFI, BspMI, EarI, FauI, FokI, HgaI, PleI, SapI, SfaNI, Sth132I, StsI.
2. Attachment of adapters with protruding ends of a suitable length and suitable type (3′ protruding or 5′ protruding end) to fragments having a protruding end, said attachment being carried out sequence-specifically. The protruding fragment ends may have been generated, in particular, by means of any of the following restriction endonucleases: AarI, AceIII, AloI, AlwI, BaeI, Bbr7I, BbsI, BbvI, BceAI, BcefI, BcgI, BciVI, BfuAI, BmrI, BplI, BpmI, BpuEI, BsaI, BsaXI, BscAI, BseMII, BseRI, BsgI, BsmAI, BsmBI, BsmFI, Bsp24I, BspCN I, BspMI, BsrDI, BstF5I, BtsI, CjeI, CjePI, EarI, EciI, Eco57I, Eco57MI, FalI, FauI, FokI, HaeIV, HgaI, Hin4I, HphI, MboII, MmeI, MnlI, PleI, PpiI, PsrI, RieAI, SapI, SfaNI, Sth132I, StsI, TaqII, TspDT I, TspGW I, Tth111II. Preferably, a plurality of adapters (“sequencing adapters”) which have different protruding ends are used in the attachment reaction. A sequential or parallel procedure in which different adapters are used in separate attachment reactions is of course also conceivable. Particular preference is given to using adapters carrying labeling groups, which differ with respect to both their protruding end and their labeling group. In one embodiment, the labeling groups are fluorophores so that, on the basis of the fluorescence activity of the attachment products, it is possible to detect which adapter has been attached to a given fragment end. The identity of the base forming a 1-base protruding end of a fragment in a mixture can be determined using, for example, adapters of the general structure
   F-Adapter-X,

with Adapter meaning the double-stranded portion of the adapter, X being any of four possible nucleotides in the form of a single-stranded protruding end and F meaning a fluorophore which characterizes the protruding base X. Thus the following assignment could be met:

| Base X | Fluorophore F |
|---|---|
| A | FAM |
| C | JOE |
| G | ROX |
| T | TAMRA |

Thus it is possible, for example, to deduce from an ROX signal obtained when fractionating the attaching products by means of an automated nucleic acid sequencer that the adapter having a protruding G was attached to a particular fragment and that, accordingly, the protruding base of the fragment in question had been a C.
Protruding fragment ends with multiple bases are usually identified “nucleotide by nucleotide”, i.e. a two-base protruding end is identified as follows: in separate mixtures, two adapters are used which have the following general structure:
   (1) F-Adapter-NX₁ for identifying the first nucleotide or
   (2) F-Adapter-X₂N for identifying the second nucleotide,
with N being a mixture of all four possible nucleotides or else a universal nucleotide such as inosine, for example. The first nucleotide of the two-base protruding fragment end would then be determined in a first reaction mixture by attaching the first adapter, and the second nucleotide of the two-base protruding fragment end would be determined in a second reaction mixture by attaching the second adapter. Preference is again given to an unambiguous and known relationship existing, as described above, between the nature of the fluorophore F and the specific nucleotide X₁ or X₂ used for sequencing. Identification of those first and second adapters which have been attached to the protruding end (usually in two parallel reaction mixtures, with the nature of the first base of the protruding end being determined in one reaction mixture and the nature of the second base of the protruding end being determined in the other mixture) can determine the sequence of said protruding end.
In a double-stranded representation, sequencing of a two-base 3′-protruding end Y₁Y₂ of a fragment is carried out according to the following diagram, for example:

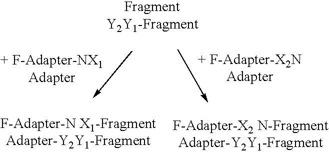

The sequence of the protruding end Y₁Y₂ can then be found in the table below:

| | 1 st Adapter | 1 st Adapter | 1 st Adapter | 1 st Adapter |
|---|---|---|---|---|
| | FAM | JOE | ROX | TAMRA |
| Y₁Y₂ | (X₁ = A) | (X₁ = C) | (X₁ = G) | (X₁ = T) |
| 2nd Adapter | TT | GT | CT | AT |
| FAM | | | | |
| (X₂ = A) | | | | |
| 2nd Adapter | TG | GG | CG | AG |
| JOE | | | | |
| (X₂ = C) | | | | |
| 2nd Adapter | TC | GC | CC | AC |
| ROX | | | | |
| (X₂ = G) | | | | |
| 2nd Adapter | TA | GA | CA | AA |
| TAMRA | | | | |
| (X₂ = T) | | | | |

Analogously, it is also possible, of course, to sequence in this way protruding ends of more than two nucleotides in length, i.e. of three or four nucleotides, for example. Furthermore, to identify more than one base of the protruding ends generated within a single experiment, labeling groups may be used which allow simultaneous detection of more than four (i.e. usually an integer multiple of four) different labels. In this case, it would be possible to use the first four of said different labels for identifying a first base of protruding fragment ends generated, the second four of said different labels for identifying a second base of the protruding fragment ends generated and, where appropriate, further sets of in each case four different labels for further bases of the protruding fragment ends generated. A “multiplexing” of this kind would result in a reduction in the number of experimental steps required. Suitable labeling groups of which numerous different ones can be detected together in one measurement, without the measured results influencing each other, would be “quantum dots”, for example (Han et al., Nat. Biotechnol. 19, 631-5 [2001]).
3. Extension of selective oligonucleotide primers whose 3′-end nucleotide or nucleotides can hybridize with the nucleotide(s) to be sequenced of the counterstrand, followed by identification of those primers which have been extended in the extension reaction. Where appropriate, said extension may be carried out by means of the polymerase chain reaction (PCR). Preference is given to firstly attaching to the ends of the nucleic acid fragments to be sequenced linkers or adapters which can serve as common primer binding sites for all or many fragments. The oligonucleotide primers are then designed so as to be able, after denaturing of the nucleic acid fragments to be sequenced, to hybridize with the linker strand attached to the 3′ end of the nucleic acid fragment strands. Care must be taken here that the oligonucleotide primers hybridized in this manner “overlap”by one or more nucleotides with the region of the nucleic acid fragment adjacent to the linker region, i.e. that they have on their 3′ end nucleotides which can hybridize with the nucleotides of said nucleic acid fragment, provided that there is complementarity. They are thus “selective nucleotides” which allow extension of the primer by means of a polymerase if they have become part of a double strand by way of said hybridization but which at least substantially prevent extension of the primer if they were unable to form a base pair with the counterstrand.

For example, in the following situation in which the selective primer 5′-YYYYYYYYYN-3′ has hybridized to the linker region XXXXXXXX of the fragment of the sequence 5′-OOOOOOOOOOOOOOOOOOOOOOOMXXXXXXXX-3′, the hybridized primer can be extended efficiently only if the selective base N of the primer is complementary to the last fragment-specific base M:

5′-YYYYYYYYYN^{␣}
3′-XXXXXXXXXMOOOOOOOOOOOOOOOOOOOOOOO-5′

The identification of in each case one or more nucleotides, which is simultaneous for a plurality of or all nucleic acid fragments, is preferably carried out after fractionating the nucleic acid fragments present in the mixture according to a fragment-specific property, in particular according to size and/or mobility of said fragments by electrophoretic fractionation. Particular preference is given to the method of gel electrophoresis in which slab gels or gel-filled capillaries are used for fractionation. In a preferred embodiment, enzymatic reactions according to variants 1-3 are carried out in step (c) in such a way that in parallel reaction mixtures in each case one or in each case two nucleotides of the fragments are identified, with said nucleotides of the fragments, to be identified in said parallel mixtures, are located in a defined position to one another, for example adjacent to one another. Then one or two nucleotides of known positions are first determined in parallel fractionations of said mixtures for each of the fragments fractionated, preferably by means of different labeling groups which allow information about the nucleotides to be determined. In a further step, the nucleotides determined for individual or all of the fractionated fragments are then put in the order in which they are present on the corresponding starting fragment from the mixture of nucleic acid fragments. The order of these two measures may of course also be reversed. In any case, signatures are generated in this way for the fragments investigated in the form of short sequence sections which characterize the corresponding fragment. The length of these sequence sections is preferably at least 14 bases, more preferably at least 16 bases, in particular at least 20 bases. Besides one or more sequence sections, a signature may also contain other information characterizing a fragment, for example accurate or approximate distances (indicated in base pairs) between characteristic regions of said fragment, for example the distance between two known sequence sections, between a known sequence section and one end of the fragment or between both fragment ends, which distance is estimated with the aid of an internal length standard on the basis of electrophoretic mobility. In this case, the sequence sections are preferably at least 10 bases in length. In any case, the information content of a signature is preferably large enough in order to allow unambiguous identification and/or isolation of the corresponding fragment. From experience, for example, approx. 14-20 base pairs of sequence information without additional information about distances within the fragment in question are usually sufficient in order to detect a transcript comprising this sequence section out of a mixture of cDNA molecules and to identify the corresponding gene. This fact is utilized, for example, by “tag-sequencing” methods such as SAGE (Velculescu et al., Science 270: 484-487 [1995], WO 00/53806) or MPSS (Brenner et al., Nature Biotechnol. 18: 630-634 [2000]). It must be taken into account here that a partial sequence for unambiguous identification of a transcript in the transcriptome usually needs to be longer than the minimum theoretical length, since the nucleotide sequence in genomes is not entirely random and particular nucleotide sequences are preferred. Accordingly, a signature consisting of a sequence of 8 nucleotides, which could theoretically code for 4⁸=65 536 different transcripts would identify in practice numerous different human cDNAs all of which would be distinguished by said signature. In contrast to this, the currently estimated number of human genes is merely approx. 30 000-40 000. Thus, in order to ensure unambiguity, the information content of a signature must sufficiently exceed the theoretical minimum. The information content of a signature characterizing a fragment can be increased inter alia by the following information:
1. a longer sequence,
2. information about actual or approximate length even of regions of the fragment, whose sequence is unknown,
3. preselection of possible identities.

When preselecting possible identities, additional information about the fragment to be identified or about the possible corresponding transcripts or genes reduces the number or probability of possible wrong assignments. Additional information about the fragment to be identified could be, for example, “3′ fragment of double-stranded cDNA generated by means of the restriction endonuclease RsaI”, which information would recognize the identity of the sequence portion of a signature with a sequence region of a transcript, which is located, viewed in 5′→3′ direction, “upstream” or “in front of” of the RsaI recognition site closest to the-3′ end of the fragment, as being insignificant. Furthermore, signatures whose sequence portion would be in the wrong orientation with respect to the preferred 5′-3′ direction of an mRNA sequence or of the cDNA sequence derived therefrom would also be identified as being insignificant. Hereto, the additional information used is the molecular-biological procedure by which the signatures have been generated, thus excluding an occurrence of particular partial sequences as signature or part of a signature. Additional information about possible genes could be, for example, “from the entirety of all genes expressed in the leaf”, if transcripts from leaf samples are to be identified by means of plant signatures generated but, for example, genes expressed exclusively in the root are not to be considered.

In a preferred embodiment of the method of the invention of analyzing nucleic acid fragments, simultaneous identification of one or more nucleotides of the cut nucleic acid fragments in step c) is carried out via the following individual steps:
ca) identifying a first nucleotide of the cut nucleic acid fragments of (b), said identification being carried out simultaneously for a plurality of or all nucleic acid fragments,
cb) identifying, where appropriate, a further nucleotide of said cut nucleic acid fragments of (b), said identification being carried out simultaneously for a plurality of or all nucleic acid fragments,
cc) repeating, where appropriate, step (cb), until the desired number of nucleotides have been identified,
cd) combining the sequence information obtained in steps (ca) to (cc) for a selected group or for all nucleic acid fragments to fragment-specific signatures, with a signature being able to contain, in addition to said sequence information, also further information about the particular fragment,
with the nucelotide identification in steps (ca) to (cc), where appropriate, additionally also comprising fractionating the nucleic acid fragments of the mixture.

In another preferred embodiment, at least one fraction of the mixture of nucleic acid fragments provided in step a) is subjected to the following method steps aa) to ad):
aa) fractionating the mixture of nucleic acid fragments according to at least one fragment-specific property,
ab) detecting, where appropriate, the relative abundance of some or all fragments in the mixture fractionated,
ac) comparing, where appropriate, the information obtained in (aa) and/or (ab) about the composition of various mixtures of nucleic acid fragments of step (a),
ad) registering, where appropriate, nucleic acid fragments detected in (ab) and/or (ac) which occur with different relative abundance in various mixtures of nucleic acid fragments,
   while a fragment mixture selected from the group consisting of I) to III) is treated according to steps b) and c), with
I) being a further fraction of the mixture of nucleic acid fragments provided in step a),
II) being a fraction of the mixture of nucleic acid fragments provided in step a) which has previously been fractionated according to at least one fragment-specific property,
III) being a mixture of nucleic acid fragments which is at least partially identical to I) or II).

Further preference is given to at least one fragment of interest of any of the groups (I) to (III) is obtained in an additional method step in any of the inventive methods above.

The fragments of interest are obtained here preferably by specific PCR amplification from a mixture of nucleic acid fragments, using fragment-specific oligonucleotide primers which can be accessed and prepared by way of the signatures determined in step (cd).

Another preferred embodiment relates to any of the inventive methods above which comprises providing a mixture of nucleic acid fragments according to step a) or a fraction of said mixture of nucleic acid fragments according to step a), either of which has been prepared by the following steps:
i) flanking of the restriction fragments of the mixture on either side by identical or different adapters;
ii) hybridizing the fragments of step (i) with in each case different primers all of which have regions complementary to the adapters of step (i) and whose 3′ end has in each case one or more nucleotides which, after hybridization of the primer with its target sequence, protrude beyond the region complementary to the adapter and which are complementary to the nucleotides of a subset of the fragments of the nucleic acid mixture of (a), which nucleotides are located opposite of said primers in the double strand.
iii) Sequence-specific extension of the primers of (ii) and, where appropriate, subsequent PCR amplification of the nucleic acid fragments of the fragment mixture, which had been extended sequence-specifically in step (ii).

Sequence-specific extension means that only, or at least primarily, those primers are extended whose nucleotide or nucleotides on the 3′ end according to step ii) is or are complementary to the nucleotides opposite thereto of the fragment with which they have formed by way of hybridization a nucleic acid double strand.

In a particularly preferred embodiment of the method of the invention, a method of gene expression analysis is provided, which comprises the following steps:
a1) providing at least one mixture of nucleic acid fragments, in particular at least wee mixture of cDNA fragments,
b1) fractionating the mixture of nucleic acid fragments according to at least one fragment-specific property,
c1) detecting, where appropriate, the relative abundance of some or all fragments in the fractionated mixture,
d1) comparing, where appropriate, the information obtained in (b1) and/or (c1) about the composition of various mixtures of nucleic acid fragments of (a1),
e1) registering, where appropriate, nucleic acid fragments detected in (d1) which appear in various mixtures of nucleic acid fragments with different relative abundances;
f1) incubating a mixture of nucleic acid fragments selected from
the group I: a fraction of the mixture of (a1),
the group II: the mixture of cDNA fragments fractionated in (b1) or a part thereof,
the group III: a mixture of nucleic acid fragments which is at least partially identical to the mixture of (a1) or to the fractionated mixture of (b1), but which additionally has at least one recognition site for a restriction endonuclease cutting outside its recognition site,
with the restriction endonuclease or restriction nucleases cutting outside its/their recognition site,
g1) identifying a first nucleotide of the cut nucleic acid fragments of (f1), said identification being carried out simultaneously for a plurality of or all nucleic acid fragments,
h1) identifying, where appropriate, a further nucleotide of the cut nucleic acid fragments of (f1), said identification being carried out simultaneously for a plurality of or all nucleic acid fragments,
i1) repeating, where appropriate, step (h1), until the desired number of nucleotides have been identified,
j1) repeating, where appropriate, once or several times steps (f1) to (i1), with the position and/or sequence of the recognition site being varied in each case in such a way that repeating once or several times allows in each case nucleotides to be identified which have not been identified previously,
k1) combining the sequence information, obtained in steps (g1) to (j1), for a selected group or for all nucleic acid fragments to give fragment-specific signatures, it being possible for a signature to contain, in addition to said sequence information, still further information about the particular fragment,
l1) where appropriate, obtaining fragments of interest from the mixture of nucleic acid fragments of (a1) or (b1), it being possible for said fragments of interest to be the fragments registered in (e),
m1) where appropriate, identifying the genes corresponding to the nucleic acid fragments of interest, from which said nucleic acid fragments are derived, by means of screening electronic databases, it being possible for said fragments of interest to be the fragments registered in (e).

When repeating steps (f1) to (i), changing the position and/or sequence of the recognition site takes care of converting other than the previously studied nucleotide positions of the fragments to be analyzed to single-stranded protruding ends and thus enabling further nucleotides not yet identified previously to be identified. Besides a sequential procedure, a simultaneous procedure in parallel approaches is of course also possible. A preferred procedure involves the following: at least one fragment mixture is provided in which many or all fragments have identical ends, for example blunt ends or protruding ends of the same length and sequence. This mixture is divided into aliquots, for example into 10 aliquots of essentially the same size. Each of the mixtures is admixed with any from a selection of different adapters (i.e. here with any of 10 different adapters) and subjected to ligation conditions, all adapters being distinguished by an end compatible to the fragment ends, i.e. attachable thereto. Furthermore, all adapters have at least one recognition site for a restriction endonuclease cutting outside its recognition sequence, for example MmeI. The adapters here differ in the distance of the recognition sequence from the adapter end to be attached to the fragment ends. In a particularly preferred embodiment, two different adapters differ in this distance by an integer multiple of the length of the protruding ends which can be generated by said restriction endonuclease cutting outside its recognition sequence. In the example of the restriction endonuclease MmeI (cutting characteristics TCCRAC(20/18)), the distance accordingly is in some adapters 18 bp, in other adapters 16 bp, in the remaining adapters 14 bp, 12 bp, 10 bp, 8 bp, 6 bp, 4 bp, 2 bp or 0 bp. If then all 10 adapter attachment products are subjected to incubation with the restriction endonuclease, in this case MmeI, thus, in the case of the first reaction, bases 19 and 20, in the second reaction, bases 17 and 18, in the remaining reactions, bases 15 and 16, 13 and 14, 11 and 12, 9 and 10, 7 and 8, 5 and 6, 3 and 4 and, respectively, 1 and 2 are exposed in the form of a single-stranded protruding end. Thus, the complete set of all 10 rations allows a contiguous partial sequence or signature of 20 bases in length for the fragments present in the fragment mixture to be identified. Apart from changing the position of a cleavage site, it would of course also be conceivable to provide at one and the same position of different adapters recognition sites for restriction endonucleases cutting at a different distance from their recognition sites. Thus, for example, an adapter could have at its end to be attached to the fragment ends a recognition site for EarI (cutting characteristics CTCTTC(1/4)), a second adapter could have at the same position a recognition site for SfaNI (cutting characteristics GCATC (5/9)) and a third adapter could have at the same position a recognition site for StsI (cutting characteristics GGATG (10/14)), thereby making it possible to identify by means of the method of the invention 13 base partial sequences of the fragments. A combination of both procedures (changing position and sequence) is also conceivable, of course.

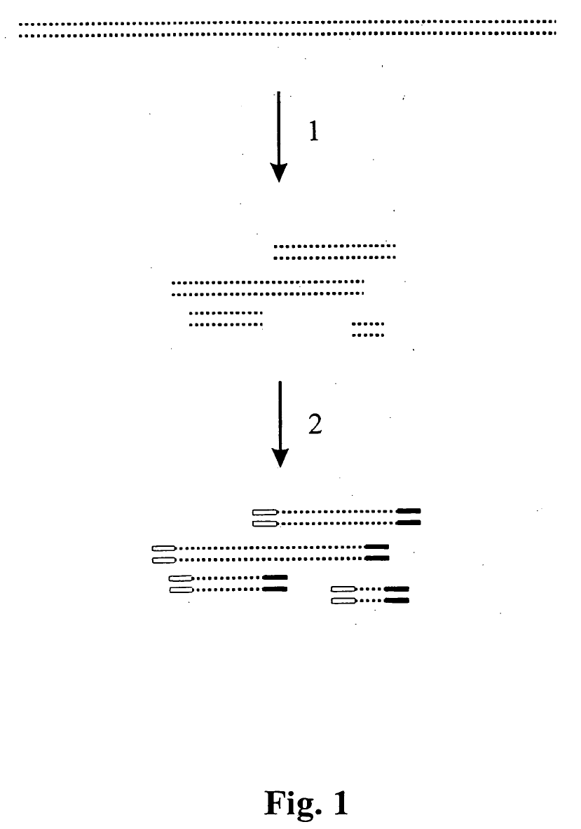

In another, particularly preferred embodiment of the method of the invention, a method of gene expression analysis is provided, which comprises the following steps:
a2) providing at least one mixture of nucleic acid fragments, in particular a mixture of cDNA fragments, having at least one recognition site for a restriction endonuclease cutting outside its recognition site, which recognition site is located on linkers added to starting fragments,
b2) incubating the mixture of nucleic acid fragments of (a2) with the restriction endonuclease or the restriction endonucleases of step (a2),
c2) identifying a first nucleotide of the cut nucleic acid fragments of (b2), said identification being carried out simultaneously for a plurality of or all nucleic acid fragments of the mixture and with fractionation of the mixture of cut nucleic acid fragments treated in a manner suitable for identifying the nucleotide, according to at least one fragment-specific property,
d2) identifying, where appropriate, a further nucleotide of the cut nucleic acid fragments of (b2) according to step (c2),
e2) repeating, where appropriate, step (d2), until the desired number of nucleotides has been identified,
f2) repeating, where appropriate, once or several times steps (a2) to (e2), with the position and/or sequence of the recognition site having been modified in each case in such a way that the repetition or repetitions allows in each case nucleotides to be identified which have not been identified previously,
g2) combining the sequence information, obtained in steps (c2) to (f2), for a selected group or all nucleic acid fragments to give fragment-specific signatures, it being possible for a signature to contain, in addition to said sequence information, still further information about the particular fragment,
h2) assigning the fragment-specific information obtained from the fractionation according to a fragment-specific property in (c2) to the signatures obtained for the nucleic acid fragments in (g2), said fragment-specific information comprising, in the case of an electrophoretic fractionation of the fragments, the relative or absolute mobility of said fragments and/or the apparent or actual fragment length determined on the basis of a length standard and it being possible for said assigning to be done in table form and/or in a computer-readable form,
i2) identifying, where appropriate, the genes corresponding to the nucleic acid fragments, from which said nucleic acid fragments are derived, by means of screening electronic databases for the signatures of (g2);
j2) providing, where appropriate, at least one further mixture of nucleic acid fragments, in particular a mixture of cDNA fragments, obtained in an analogous way to the mixture of nucleic acid fragments of (a2), it being possible here to dispense with the adding of linkers having at least one recognition site for a restriction endonuclease cutting outside its recognition site,
k2) fractionating the mixture or mixtures of nucleic acid fragments of (i2) according to a fragment-specific property, essentially under the conditions of the fractionation in (c2),
l2) assigning the fragment-specific information obtained from the fractionation according to a fragment-specific property in (k2) to the individual fractionated fragments, it being possible for said fragment-specific information to comprise relative or absolute abundance of the individual fragments and also, in the case of electrophoretic fractionation of the fragments, the relative or absolute mobility of said fragments and/or the apparent or actual fragment length determined on the basis of a length standard and the assignment to be carried out in table form and/or in a computer-readable form, m2) comparing, where appropriate, the relative or absolute abundances of at least part of the fragments fractionated in (k2) to the relative or absolute abundances of in each case homologous i.e. completely or essentially sequence-identical, fragments derived from various mixtures of nucleic acid fragments,
n2) registering, where appropriate, those fragments whose relative or absolute abundance differs from the relative or absolute abundance of their homologous fragments of other mixtures of nucleic acid fragments by at least one preselected factor,
o2) assigning, where appropriate, the fragments registered in (n2) to those genes or transcripts from which said fragments are derived, using the results obtained in step (h2),
p2) obtaining, where appropriate, the fragments registered in (n2) from the mixture of nucleic acid fragments of (a2) or (i2) and/or j2),
it also being possible for steps (i2) to (n2) to be carried out before steps (a2) to (h2).
Mixtures of nucleic acid fragments, preferably mixtures of cDNA fragments, may be generated by methods known from the prior art. For example, EP 0 743 367, which is hereby incorporated by reference in its entirety, describes the generation of fragments obtained by means of usually frequently cutting restriction endonucleases, which represents the 3′ ends of cDNA molecules and are flanked on one side by linkers and which are amplified by means of selective PCR primers (extended on their 3′ end beyond the “universal” binding site common to all primers of one type by one or more “selective” nucleotides) in the form of a plurality of subgroups (“subpools”). Each of these subgroups then comprises a subset of the initially generated entirety of all cDNA 3′ fragments. Fragment subpools obtained from various RNA preparations to be studied for differentially expressed genes, which subpools correspond to one another (i.e. have been generated using the same selective primers), are then fractionated according to their size by way of gel electrophoresis, and the band or signal patterns obtained are compared to one anther. Bands or signals coming from homologous fragments, whose intensity differs between different samples, represent genes whose level of expression differs in the samples compared (cf. for example, FIG. 1 of EP 0 743 367). Other, alternative methods of generating mixtures of cDNA fragments for expression analysis are known from the prior art, cf., for example, Kato: Nucleic Acids Res. 23, 3685-3690 (1995), Ivanova et al., Nucleic Acids Res. 23, 2954-2958 (1995), Bachem et al, Plant J. 9, 745-753 (1996), Prashar et al., Proc. Natl. Acad. Sci. U. S. A. 93, 659-663 (1996), Shimkets et al., Nat. Biotechnol. 17, 798-803 (1999), Ke et al., Analyt. Biochem. 269, 201-204 (1999)., Jing et al., Analyt. Biochem. 287, 334-337 (2000), Sutcliffe et al., Proc. Natl. Acad. Sci. U S. A. 97, 1976-1981 (2000), WO 99/42610, EP 0 981 609.

The fragment-specific property is a, in particular physical or physicochemical, property which may be realized by various molecules within a continuum or in the form of a relatively large number (e.g. at least 10 or at least 100) of different grades or phenotypes. Particular preference is given to utilizing different mobilities of different nucleic acid fragments in separation systems, in particular different electrophoretic mobility in electrophoretic systems such as agarose or polyacrylamide gel electrophoresis. Here, said mobility is usually influenced by the length of a fragment; however, this is not a strictly linear relationship, since G/C content and conformation of a nucleic acid molecule also influence mobility. Therefore, the mobility of a nucleic acid molecule can usually be used for determining only the approximate but not the absolute size. Furthermore, said fragment-specific property may be a particular partial sequence of n nucleotides, where n may be equal to or greater than 1. Preferably, said partial sequence of a fragment is adjacent to a linker attached to the end of said fragment so that a mixture of different fragments can be fractionated according to this partial sequence via extension, where appropriate a repeated extension in the form of amplification, of selective oligonucleotide primers. A procedure of this kind is described in EP 0 743 367, for example. In this case, “fractionating a fragment mixture” means the preparation of mixtures of amplified fragments, each of which contains copies generated by amplification of only a part of the fragments present in the starting mixture. In another preferred case, said partial sequence is at least partially in the form of a single-stranded protruding end, and a mixture of different fragments is fractionated according to said partial sequence via attachment of adapters having compatible protruding ends. This process, also referred to as “categorizing of nucleotide sequence populations”, is described in WO 94/01582. A combination of both measures is also conceivable and described, for example, in WO 01/75180.

Detection of the relative abundance of some or all fragments is carried out by way of measuring the signal strength obtained in the detection of individual nucleic acid fragments. In a preferred embodiment, the nucleic acid fragments contain detectable labeling groups, particular preference being given to using fluorophores as labeling groups. If, for example, an automated sequencer is used for fractionation and detection, ten the relative abundance of a fragment can be readily obtained as the area under the corresponding curve in a fluorogram (plotting of the measured fluorescence intensity as a function of the retention time) in the form of a number. A fragment here means the entirety of all sequence-identical nucleic acid molecules of a mixture, where appropriate with addition of the nucleic acid molecules having a sequence complementary thereto. The numbers obtained as relative abundances of fragments are often stored in a computer-readable form.

In the step of registering nucleic acid fragments, preferably cDNA fragments, of different relative abundances, those fragments are identified whose proportion differs between different biological samples or between different mixtures of cDNA fragments. If care is taken to generate from the mRNA molecules present in said samples cDNA fragments whose abundance distribution is similar or even equal to the abundance distribution of the different mRNA molecules, then cDNA fragments of different abundance between fragment mixtures that are compared to one another also indicate mRNA molecules of different abundance and thus differentially expressed genes. In order to compensate for relatively small fluctuations, for example in the efficiency of the enzymatic steps carried out before or of detection, it is possible, where appropriate, to determine a threshold for abundance differences so that, for example, only those cDNA fragments are studied further whose relative abundance between fragment mixtures compared to one another differs by at least a factor of two.

Simultaneous identification of a nucleotide or of a plurality of nucleotides for a plurality of or all nucleic acid fragments is preferably conducted by carrying out, as described above, a process characteristic for the identity of the nucleotide to be identified in each case on protruding fragment ends generated by means of at least one restriction endonuclease cutting outside its recognition site, for which process a mixture of a plurality of or all nucleic acid fragments is used and whose result can be observed preferably via incorporation of a label, in particular a fluorescent label. Preference is given here to the identified nucleotides being adjacent to one another, i.e. the information thus obtained about the nucleotide identities resulting in a contiguous partial sequence of the particular nucleic acid fragment. In a preferred embodiment, said process, the “sequencing reaction”, is followed by a fractionation of the products produced in said process, it being possible here for said fractionation to be carried out again according to the fragment-specific property of (b1) or (c2).

Combining the sequence information obtained to give fragment-specific signatures involves assigning to each or some of the fractionated nucleic acid molecules the nucleotide identity obtained for some positions. The information obtained about a fragment is referred to as signature. Said signature here can, besides sequence information, contain still further information, for example sequence information obtained in a different way or the approximate fragment size obtained via fragment mobility. If, for example, 3′cDNA-fragments are generated using the restriction endonuclease RsaI (recognition sequence GTAC), according to EP 0 743 367 mentioned above, and if, a selected fragment, the identity A (1st nucleotide), G (2nd nucleotide), T (3rd nucleotide), and A (4th nucleotide) is assigned to the nucleotides identified in steps (g1) to (j1), as viewed from the recognition site for RsaI, then it is possible to generate therefrom a sequence signature of the nucleotide sequence GTACAGTA. Further secondary information which could also be included, in addition to the approximate fragment size, is the fact that no other identical partial sequence can be located by nature between the partial sequence GTAC and the 3′ end of the fragment (provided that the RsaI digestion has been completed). In any case, fragment-specific signatures can be determined for all or for part of the fragments obtained in a fragment mixture. When applying the method of the invention to comparative gene expression analysis, signatures are determined in particular for those fragments which differ in their relative abundance between the fragment mixtures to be compared by at least one specified factor.

Incidentally, the sequence portion of a signature need not necessarily be a contiguous sequence. Thus it is conceivable, for example, that terminal nucleotide partial sequences of both fragment ends of a given fragment are determined and combined to give a signature; here too, it is of course possible to include further information into the signature, such as, for example, approximate fragment length. For example, the signature

5′-CTCA{192}GGAT-3′

could mean for a particular fragment that said fragment “starts” at the 5′ end with the nucleotide sequence CTCA, “stops” at the 3′ end with the nucleotide sequence GGAT and has a total length, where appropriate with additional terminal linker regions, of approximately 200 bp (=4 bp +192 bp +4 bp). Here, the phrase “approximately” takes into account that the determination of fragment length on the basis of electrophoretic mobility is subject to a certain error, as discussed above.

Fragments of interest may be obtained from the mixture of nucleic acid fragments, preferably of cDNA fragments, for example by means of PCR with the aid of gene-specific primers and with the help of the fragment-specific signatures determined. If, for example in the example above, a mixture of 3′ cDNA fragments has been obtained by means of the restriction endonuclease RsaI, followed by the ligation of linkers to the (blunt) fragment ends generated, and if the above signature GTACAGTA has been obtained for a selected fragment, then the information about the fragment is that, after RsaI cleavage (removing, inter alia, the first two nucleotides of the RsaI ligation site, GT), the first nucleotides following the linker sequence have the sequence ACAGTA. If a primer is then used for PCR amplification, which has the very nucleotide sequence ACAGTA following the linker sequence at its 3′ end, then the corresponding fragment is directly accessible by amplification from the fragment mixture, since said primer selectively promotes amplification of those fragments whose sequence is identical (or complementary) to its own over its entire length. The fragment thus obtained may then be subjected to further analysis, for example sequencing, followed by a database query for entries with identical or similar sequences. This procedure requires of course a sufficiently high information content of the signature, i.e. a sufficient length and thus specificity of the fragment-specific region of the amplification primer. Were the partial sequence ACAGTA to be directly adjacent to the linker region in more than one of the fragments present in the mixture, then it would be possible to amplify a mixture of these fragments with the help of said primer. In order to obtain an individual fragment of interest in the manner described, the primer used would therefore have to be extended at its 3′ end by further specific bases. In this case, it must also be taken into account that the ability of polymerases to discriminate against the extension of primers hybridized to the template strand with partial mismatch is reduced with increasing distance of said mismatches from the 3′ end of the primer. If a primer is thus extended at its 3′ end by further fragment-specific bases to increase specificity, a certain loss of specificity can be expected for those bases which are immediately downstream of the sequence section of the primer, which is complementary to the particular linker sequence.

In a preferred application of the method of the invention, the signatures obtained for nucleic acid fragments of interest are used for designing fragment-specific oligonucleotide primers. In this application, preference is furthermore given to using the oligonucleotide primers obtained for amplifying selected fragments, usually employing the mixture of nucleic acid fragments or a fraction thereof as amplification template.

Identification of the genes associated with the nucleic acid or cDNA fragments of interest may be carried out by means of screening electronic databases, if the information content of a signature is large enough in order to permit unambiguous or substantially unambiguous identification of a gene and if the database has relevant entries. How large the information content of signatures of a biological species must be in order to allow unambiguous assignability of a signature to the corresponding gene, must be determined empirically and may be different from gene to gene, even within a biological species; thus it may happen that a particular decamer (a signature consisting of 10 nucleotides) is characteristic for a single gene, while a different decamer appears in numerous different genes.

In a preferred application of the method of the invention, the signatures obtained for nucleic acid fragments of interest are used for identifying said nucleic acid fragments in a database search.

In another preferred application of the method of the invention, the signatures obtained for nucleic acid fragments of interest are used for generating EST libraries. To this end, the signatures obtained for the individual fragments obtained from a cDNA preparation are used in order to design fragment-specific oligonucleotide primers which are then used to obtain the particular fragments by means of PCR amplification. The fragments obtained are finally sequenced and the sequences are recorded in a database. EST libraries generated in this way may also be referred to as normalized EST libraries, since each fragment is generated only once, independently of its abundance or of the abundance of the mRNA or cDNA molecules which it represents. This is of great advantage in comparison with the EST libraries generated according to the prior art which exhibit an extremely high degree of redundance (cf. Lee et al., Proc. Natl. Acad. Sci. U.S.A. 92, 8303-8307 [1995]). Said reduncance of EST libraries prepared in the traditional way results from the fact that abundant transcripts (for example of an abundance of 1000 mRNA-copies per cell) are represented by substantially more cDNA clones contributing to the EST library than less abundant transcripts (for example of an abundance of 1 mRNA-copy per cell—in this example, the frequency difference of clones representing these two transcripts would be 1:1000). The prior art furthermore discloses methods of normalizing cDNA libraries, which involve normalizing the concentration of abundant and less abundant clones by utilizing the reassociation kinetics of nucleic acids (Soares et al., Proc. Natl. Acad. Sci. U.S.A. 91, 9228-9232 [1994]). Although such normalized libraries are distinguished by a reduction in the concentration of particularly abundant clones, the difference in abundance of frequent and less frequent clones is still considerable and may be between one and two orders of magnitude, making preparation and analysis of libraries of this kind very expensive. When preparing normalized libraries according to the method of the invention, a redundance can practically be ruled out; nevertheless, in contrast to normalized libraries according to the prior art, there is no loss of information on the abundance of the individual fragments, clones and of those transcripts from which the former are derived. Rather, information on abundance can be obtained from the particular signal strength, obtained, for example, by means of fractionation via capillary gel electrophoresis, of the individual fragments of an investigated fragment mixture and added as retrievable additional information to each EST sequence obtained.

In another preferred application of the method of the invention, the mixtures of nucleic acid fragments used are mixtures of restriction fragments generated from genomic DNA or cDNA and flanked on both sides by identical or different adapters, with the adapter-flanked fragments first being subjected to an amplification by means of primers extended on their 3′ end by one or more nucleotides beyond the region complementary to the adapter and using the amplification products obtained in this way for carrying out said method.

In another embodiment of the method of the invention, the mixture of nucleic acid fragments used comprises those fragments which have been generated from genomic DNA or cDNA by restriction digestion with restriction endonucleases belonging, at least partially, to the type IIs and which are flanked on one side or on both sides by adapter sequences. In this application, the type IIs restriction endonuclease(s) generates (generate) protruding ends whose sequence is not determined directly by the restriction endonuclease but by the nucleic acid sequence of the cleavage site and which may consequently be different from fragment to fragment. If desired, adapters may be used for attachment, which can be attached only to particular protruding ends, in particular to those whose nucleotide sequence is complementary to the nucleotide sequence of the protruding adapter ends. In this way it is possible to attach particular preselected adapters only to a part of all nucleic acid fragments and thus to generate a subset of the mixture of nucleic acid fragments used (“molecular indexing”, cf. Kato, Nucleic Acids Res. 1996, January 15, 24 (2): 394-395, and WO 94/01582).

In a particularly preferred embodiment, the required enzymatic reaction mixtures are prepared by means of an automated pipetter.

In another particularly preferred embodiment, the fluorograms obtained by means of gel electrophoresis, preferably by means of capillary gel electrophoresis, are evaluated automatically. This evaluation involves assigning to one another by means of a computer system signals belonging to one another of various fluorograms which represent (i) homologous fragments of various mixtures of nucleic acid fragments, (ii) fragments of a nucleic acid mixture and the reaction products obtained for identification of one or more nucleotides of the fragment of said mixture, (iii) reaction products obtained for identification of a plurality of nucleotides of the fragments of a mixture of nucleic acid fragments. An automated assignment of this kind may be carried out, for example, according to the following protocol:
1. Select a suitable start signal which has not yet been assigned,
2. Search for the signal best fitting thereto, with the criteria being (a) as small a difference as possible in the determined fragment length and (b) as small a difference as possible in signal intensity and it being possible for these two criteria to be introduced with freely choosable weighting,
3. Repeat step (2), comparing each additional signal with the average of fragment length and signal intensity of all previously assigned signals,
4. Stop the process, when the differences of (2) exceed a preselected threshold.
5. Repeat steps (1) to (3) until all signals of a set of fluorograms to be assigned to one another have been assigned to one another or have been found to be not assignable to one another.

Furthermore, preference is given to the automated evaluation comprising carrying out the steps (d1), (e1), (g1), (h1), (i1), (j1), (k1), (m1), (c2), (d2), (e2), (f2), (g2), (h2), (i2), (l2), (m2), (n2) and/or (o2).

The invention is illustrated in more detail below by the drawings in which

FIG. 1: shows the generation of adapter-flanked nucleic acid fragments,

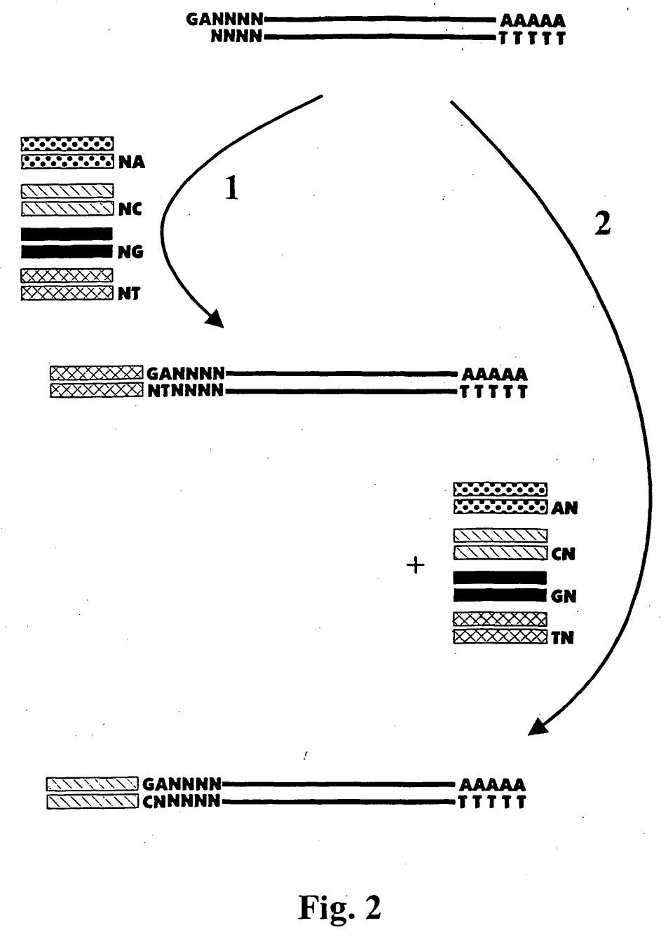

FIG. 2: shows the sequencing of protruding fragment ends by means of adapter ligation,

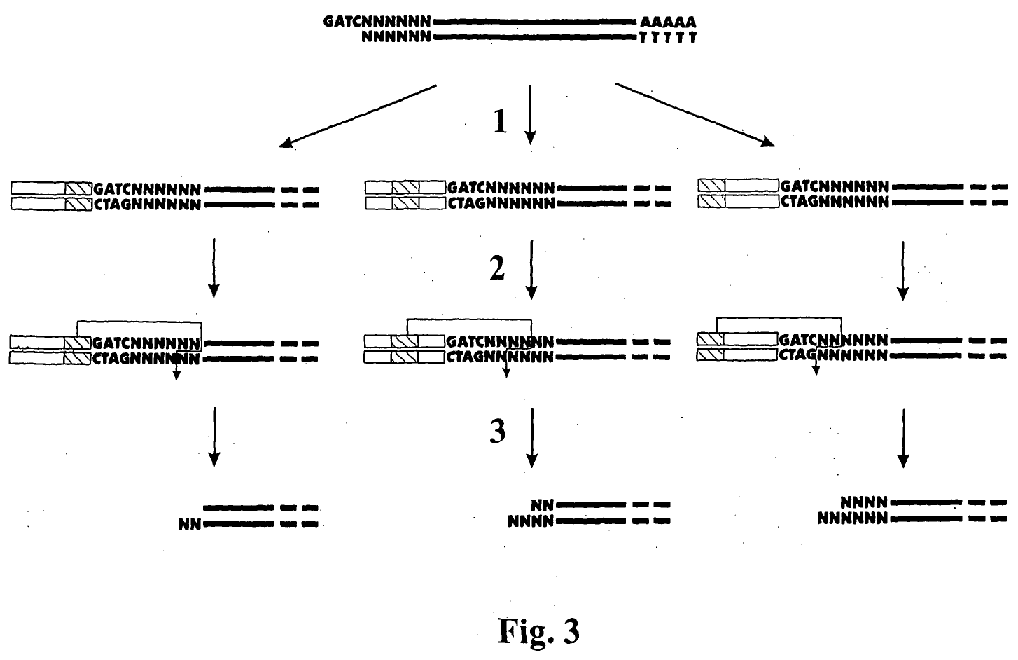

FIG. 3: shows the generation of various protruding ends by truncating a nucleic acid fragment,

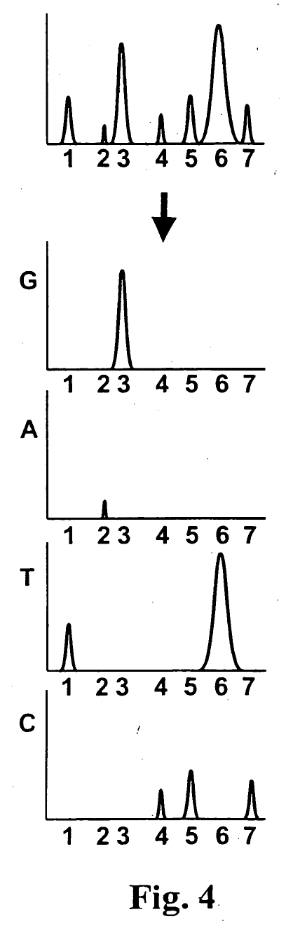

FIG. 4: shows the identification of a nucleotide for all fragments of a mixture of nucleic acid fragments,

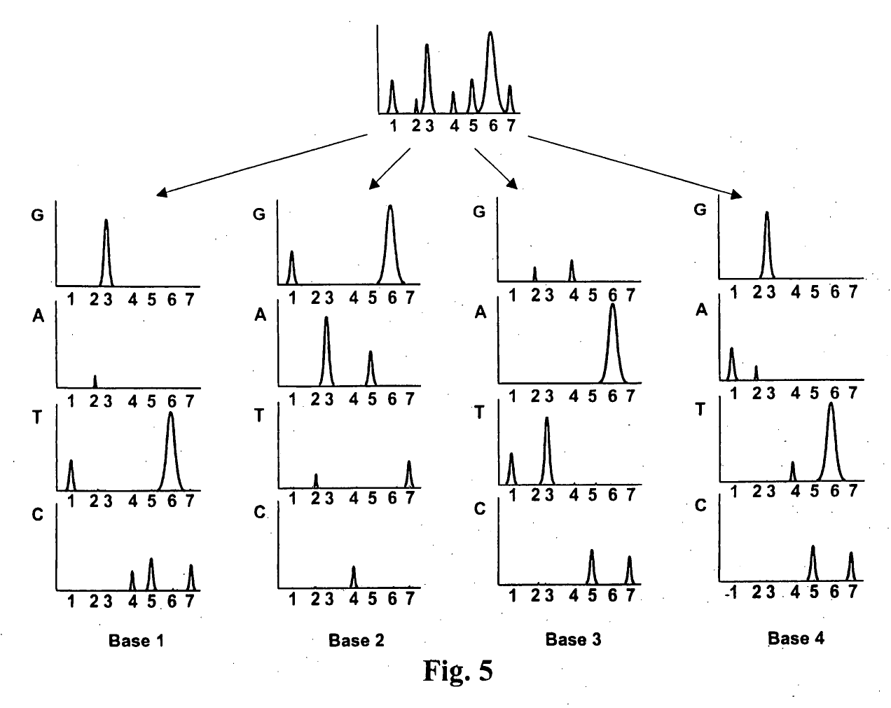

FIG. 5: shows the identification of four nucleotides for all fragments of a mixture of nucleic acid fragments.

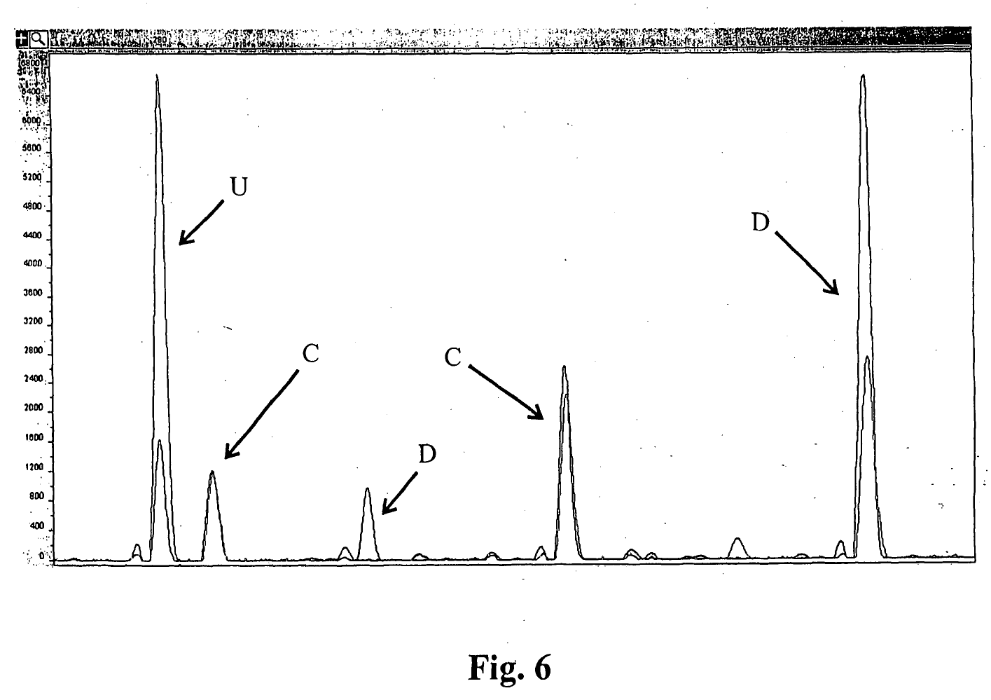

FIG. 6: shows the fractionation of a mixture of nucleic acid fragments by means of capillary gel electrophoresis,

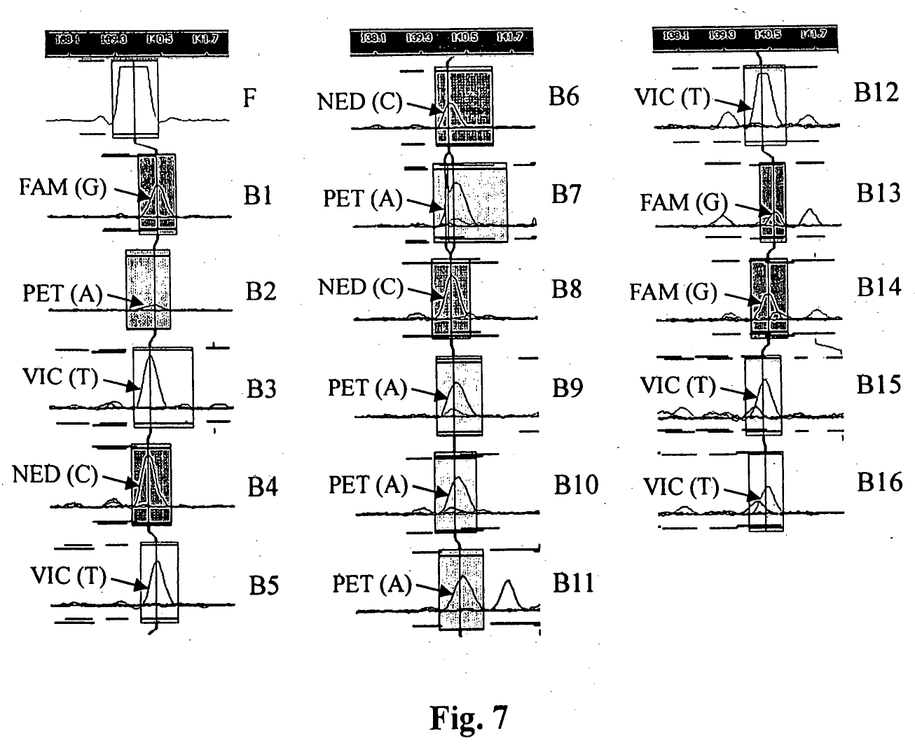

FIG. 7: the identification of a plurality of nucleotides of a nucleic acid fragment by means of capillary electrophoresis,

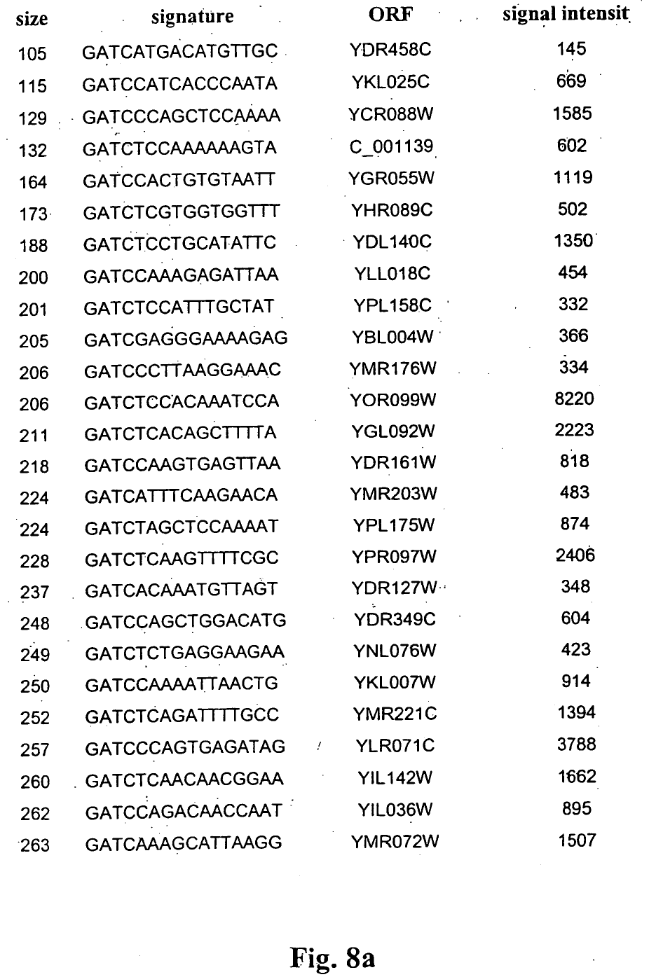

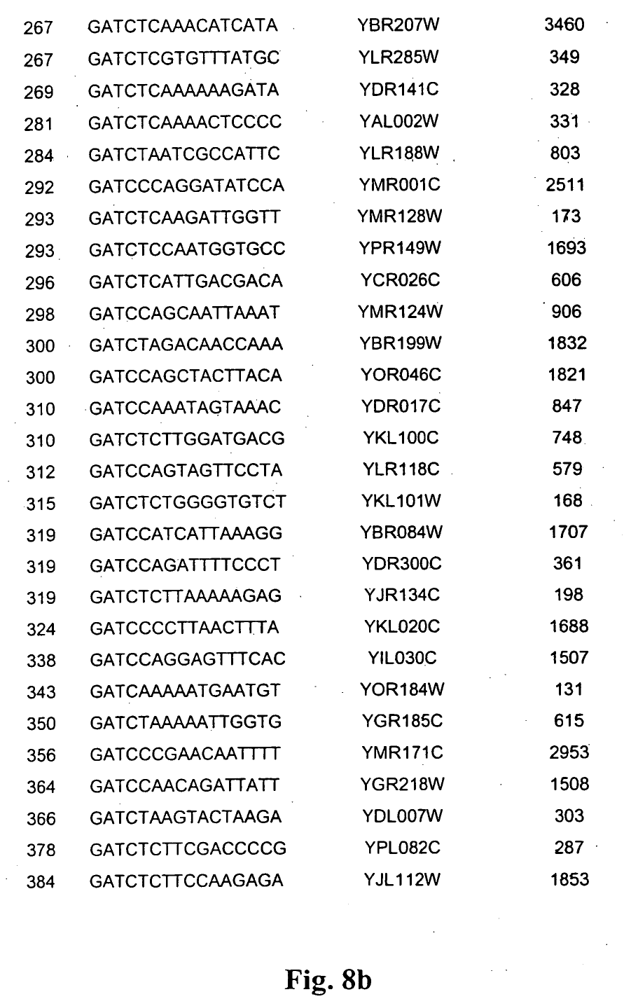

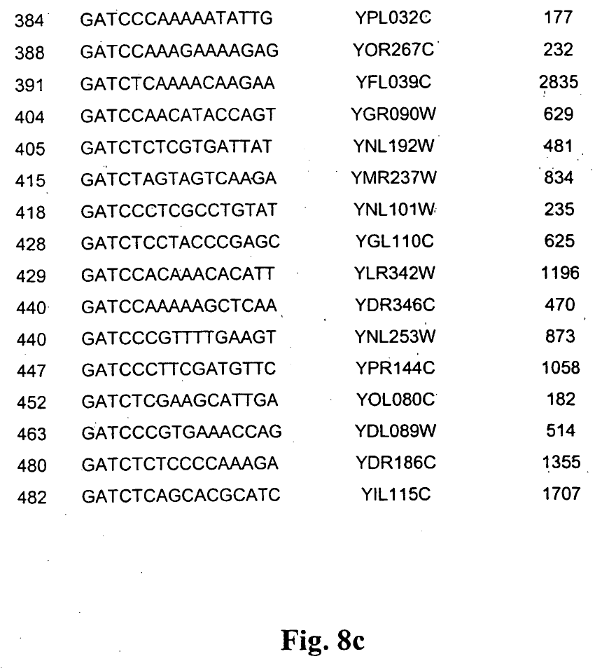

FIG. 8: shows a list of some signatures obtained from a suspension culture of Saccharomyces cerevisiae.

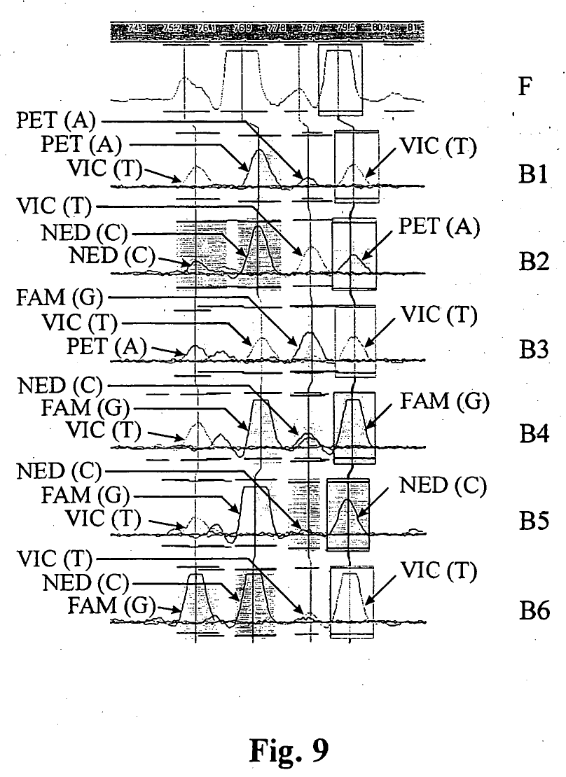

FIG. 9: shows the identification of a plurality of nucleotides of four nucleic acid fragments of a mixture of nucleic acid fragments.

FIG. 1 shows the generation of adapter-flanked nucleic acid fragments, with
1 depicting the fragmentation of a nucleic acid preparation by means of two restriction endonucleases, and
2 depicting the attachment of adapters to the fragment ends.

FIG. 2 shows the sequencing of protruding fragment ends by means of adapter ligation, with
1 showing the sequencing of the first position of said protruding ends, and
2 showing the sequencing of the second position of said protruding ends.
The sequencing of a nucleic acid fragment representing the 3′ end of a cDNA molecule is shown. The adapters used for sequencing are distinguished by a different sequence of the protruding ends and by various labeling groups which code for the sequence of the particular protruding end. A labeling group indicating the base A is indicated by a dotted adapter, a label indicating a C is indicated by a hashed adapter, a label indicating a G is indicated by a filled-in adaptor and a label indicating a T is indicated by a cross-hashed adapter. A T-indicating labeling group attached to the fragment by ligation in (I) indicates that the first base of the protruding end is the base A which is complementary thereto. A C-indicating labeling group attached to the fragment by ligation in (2) indicates that the second base of the protruding end is the base G which is complementary thereto.

FIG. 3 indicates the generation of various protruding ends by truncating a nucleic acid fragment, with
1 showing the attachment of three different adapters containing in each case in a different position a recognition site (hashed region) for a type IIS restriction endonuclease,
2 showing the incubation of the attachment products with said type IIS restriction endonuclease, and
3 showing the release of truncated protruding fragment ends which comprise, with respect to the double-stranded region of the starting fragment, the positions -5 and -6 (left), -3 and -4 (center) and -1 and -2 (right) in a terminally single-stranded form which is thus accessible to sequencing via adapter ligation.
The starting fragment depicted here is a 3′ cDNA-fragment obtained by means of the restriction endonuclease MboI.

FIG. 4 describes the identification of a base for all fragments of a mixture of nucleic acid fragments. The fragments are provided with fluorescent labeling groups and fractionated according to their mobility by means of capillary gel electrophoresis. The resulting fluorogram (depicted at the top) is used for cataloging said fragments (allocation of serial numbers). This is followed by identifying, for the position to be determined of the fragments according to the description above, the nucleotides located there. After carrying out the appropriate reactions in which the identity of said nucleotides is encoded by means of introducing nucleotide-specific labeling groups, the products are likewise fractionated by means of capillary gel electrophoresis and the identity of the labeling groups introduced is determined, taking into account mobility and, where appropriate, signal intensity. Identification of the base of interest results in a “G” for fragment 3, “A” for fragment 2, “T” for fragments 1 and 6 and “C” for fragments 4, 5 and 7.

FIG. 5 indicates the identification of four nucleotides for all fragments of a mixture of nucleic acid fragments (fragments 1-7). In the case of the sequence of the four nucleotides being contiguous, the following sequence signatures arise:

Fragment 1: TGTA Fragment 2: ATGA

Fragment 3: GATG

Fragment 4: CCGT

Fragment 5: CACC

Fragment 6: TGAT

Fragment 7: CTCC

FIG. 6 depicts the fractionation of a mixture of nucleic acid fragments by means of capillary gel electrophoresis. cDNA fragments were generated, as described, from a suspension culture of Saccharomyces cerevisiae. The signals obtained from a stationary phase (gray) and from a culture in the logarithmic phase (black) are shown. Some of the fragments represent constitutively expressed genes (signals indicated by “C”), others represent genes downregulated in the stationary phase (signals indicated by “D”) and others again represent genes upregulated in the stationary phase (signal indicated by “U”). The horizontal scale shows the fragment size, the vertical scale indicates the fluorescence intensity.

FIG. 7 shows the identification of a plurality of nucleotides of a nucleic acid fragment by means of capillary gel electrophoresis. F, one of the fragments of a mixture of nucleic acid fragments, B1-B16, identification of the first to sixteenth base of the fragment, FAM, PET, VIC, NED, the particular fluorophore detected in the identification of a base, (G), (A), (T), (C), the base identified by means of a particular fluorophore. The signature GATCTCACAAATGGTT is produced for the selected fragment. The bar at the top shows the fragment size, i.e. the fragment has a size of approximately 140 bp.

FIG. 8 shows a list of some of the signatures obtained from a suspension culture of Saccharomyces cerevisiae. Indicated in each case are the fragment size, the signatures determined according to the method of the invention, the open reading frames (ORFs) identified by means of BLAST analysis and the signal intensity obtained by means of capillary gel electrophoresis.

FIG. 9 indicates the identification of a plurality of nucleotides of four nucleic acid fragments of a mixture of nucleic acid fragments. The fragments have an approximate length of 75 bp, 77 bp, 78 bp and 79 bp. F, fractionated fragments of the mixture, B1-B6, identification of the first to sixth base of the fragments, FAM, PET, VIC, NED, the particular fluorophore detected in the identification of a base, (G), (A), (T), (C), the base identified by means of the particular fluorophore. The signature produced for the 75 bp fragment is TCATTG, the signature produced for the 77 bp fragment is ACTGGC, the signature produced for the 78 bp fragment is ATGCCT, and the signature produced for the 79 bp fragment is TATGCT.

The invention is furthermore illustrated in more detail by the following examples.

### EXAMPLE 1

### Obtaining cDNA 3′ Restriction Fragments

25 μg of total RNA from a suspension culture of Saccharomyces cerevisiae were precipitated with ethanol and dissolved in 15.5 μl of water. 0.5 μl of 10 μM cDNA primer CP31V (5′-ACCTACGTGCAGATTTTTTTTTTTTTTTTTTV-3′, SEQ ID NO: 1) was added, and the mixture was denatured at 65° C. for 5 minutes and placed on ice. 3 μl of 100 mM dithiothreitol (Life Technologies GmbH, Karlsruhe, Germany), 6 μl of 5× Superscript buffer (Life Technologies GmbH, Karlsruhe, Germany), 1.5 μl of 10 mM dNTPs, 0.6 μl of RNase inhibitor (40 U/μl; Roche Molecular Biochemicals) and 1 μl of Superscript II (200 U/μl, Life Technologies) were added to the mixture which was then incubated for cDNA first strand synthesis at 42° C. for 1 hour. For second strand synthesis, 48 μl of second strand buffer (cf. Ausubel et al., Current Protocols in Molecular Biology (1999), John Wiley & Sons), 3.6 μl of 10 mM dNTPs, 148.8 μl of H₂O, 1.2 μl of RNaseH (1.5 U/μl, Promega) and 6 μl of DNA Polymerase I (New England Biolabs GmbH, Schwalbach, Germany, 10 U/μl) were added and the reaction incubated at 22° C. for 2 hours. This was followed by extracting with 100 μl of phenol, then with 100 μl of chloroform and precipitating with 0.1 volume of sodium acetate pH 5.2 and 2.5 volumes of ethanol. After centrifugation at 15 000 g for 20 minutes and washing with 70% ethanol, the pellet was dissolved in a restriction mixture comprising 15 μl of 10× Universal buffer, 1 μl of MboI and 84 μl of H₂O, and the reaction was incubated at 37° C. for 1 hour. After extracting, first with phenol, then with chloroform, and precipitating with ethanol, the pellet was dissolved in a ligation mixture comprising 0.6 ∥l of 10× ligation buffer (Roche Molecular Biochemicals), 1 μl of 10 mM ATP (Roche Molecular Biochemicals), 1 μl of ML2025 linker (prepared by hybridization of oligonucleotides ML20 (5′-TCACATGCTAAGTCTCGCGA-3′, SEQ ID NO: 2) and LM25 (5′-GATCTCGC GAGACTTAGCATGTGAC-3′, SEQ ID NO: 3)), 6.9 μl of H₂O and 0.5 μl of T4 DNA ligase (1 U/μl; Roche Molecular Biochemicals), and ligation was carried out at 16° C. overnight. The ligation reaction was diluted with water to 100 μl, extracted with phenol, then with chloroform, and, after addition of 1 μl of glycogen (20 mg/ml, Roche Molecular Biochemicals), precipitated with 100 μl of 28% polyethylene glycol 8000 (Promega)/10 mM MgCl₂. The pellet was washed with 70% ethanol and taken up in 40 μl of water.

### EXAMPLE 2

### Amplification of cDNA 3′ Restriction Fragments with Distribution to Subpools

For the first round of amplification, PCR mixtures were prepared, comprising 2 μl of precipitated ligation reaction of Example 1, 2 μl of 10× PCR buffer (670 mM Tris-Cl, pH 8.8, 170 mM (NH₄)₂SO₄, 1% (v/v) Tween 20), 1.5 μl of 20 mM MgCl₂, 0.4 μl of 10 mM dNTPs, 2 μl of RediLoad (Invitrogen GmbH, Karlsruhe, Germany), 0.2 μl of Taq DNA polymerase (Roche Molecular Biochemicals), 1 μl of 4 μM oligonucleotide primer CP31X₁X₂ (5′-ACCTACGTGCAGA TTTTTTTTTTTTTTTTTTX₁X₂-3′ where X₁=G, A or C, X₂=G, A, T or C; SEQ ID NO: 4), 1 μl of 4 μM oligonucleotide primer ML20 and 9.9 μl of water. All 12 reactions (comprising in each case one of the 12 possible CP31X₁X₂-primers as primer) were subjected to 25 amplification cycles consisting in each case of the phases denaturation (30 sec. 94° C.), attaching (30 sec. 65° C.) and extension (2 min. 72° C.). In each case 5 μl of the reactions were checked by means of electrophoresis through a 1.5% strength agarose gel. The reactions were. diluted with water to 100 μl. Further PCR mixtures were prepared, comprising 2 μl of diluted amplification reaction, 2 μl of 10× PCR buffer, 1.5 μl of 20 mM MgCl₂, 0.4 μl of 10 mM dNTPs, 2 μl of RediLoad, 0.2 μl of Taq DNA polymerase, 1 μl of 4 μM oligonucleotide primer CP31VNX₃X₄ (5′-ACCTACGTGCAGATTTTTTTTTTTTTTTTTT VNX₃X₄-3′ where V=mixture of G, A and C, N=mixture of G, A, T and C, X₃, X₄=G, A, T or C; SEQ ID NO: 5), 1 μl of 4 μM oligonucleotide primer ML20 and 9.9 μl of water. Depending on intended further processing of the reaction mixtures, primer ML20 had a fluorescent label (selected from any of the dye sets 5′-FAM, 5′-JOE, 5′-ROX and 5′-TAMRA [dye set 1] or 5′-FAM, 5′-VIC, 5′-NED and 5′-PET [dye set 2]; further processing of the samples according to example 3), or ML20 was used in unlabeled form (further processing of the samples according to example 4 and, respectively, example 5). All 2×192 reactions (comprising in each case one of the 12 possible diluted amplification reactions and one of the 16 possible CP31VNX₃X₄ primers; 12×16=192; ML20 in each case labeled or unlabeled) were subjected to 25 amplification cycles consisting in each case of the phases denaturation (30 sec. 94° C.), attaching (30 sec. 65° C.) and extension (2 min. 72° C.). Again, in each case 5 μl of the reactions were checked by means of agarose gel electrophoresis. The remaining reaction mixtures were purified by means of QiaQuick columns (Qiagen AG, Hilden, Germany) according to the manufacturer's information; the elution was carried out in 50 μl of water in each case. The amount was determined by spectrophotometry.

### EXAMPLE 3

### Fractionation and Preparation of the Fluorescently Labeled Amplification Products by Means of Capillary Gel Electrophoresis

In each case 2 μl of the purified fluorescently labeled amplification products of example 2 were diluted with 10 μl of water and (if dye set 2 was used, after addition of 0.5 μl of GeneScan 500 LIZ length standard [Applied Biosystems GmbH, Weiterstadt, Germany]) fractionated via capillary gel electrophoresis by means of an ABI Prism 3100 Genetic Analyzer (Applied Biosystems). In order to achieve higher throughput of the instrument by “multiplexing”, further reaction mixtures were prepared by mixing in each case 1 μl of FAM-labeled amplification products, 1 μl of VIC-labeled amplification products, 1 μl of NED-labeled amplification products and 1 μl of PET-labeled amplification products, adding 0.5 μl of LIZ length standard and 7.5 μl of water and said reaction mixtures were used in electrophoresis. “Multiplexing” using dye set 1 was carried out analogously; in this case, fragments labeled with FAM, JOE or TAMRA were mixed with GeneScan 500 ROX length standard. The fluorograms were depicted and evaluated by means of GeneScan software, version 3.7 for Windows NT (Applied Biosystems). Differentially expressed genes were identified by comparing fluorograms to one another which had been obtained from RNA preparations of yeast cells in various growth stages but using the same amplification primers of the first and the second rounds of amplification. To this end, the fluorograms were superimposed by means of GeneScan and visually studied for differences in the signal patterns obtained. For comparisons of this kind, care was first taken, by means of the GeneScan function “align data by size”, that it was possible to assign to one another fragments “matching” each other (i.e. representing the same gene/transcript) from RNA preparations of different growth stages. In the next step, the signal strengths were normalized by adjusting the average height of the signals of a sample to the average signal strength of a sample to be compared therewith. Differentially expressed genes were identified by listing signals which appear in samples compared to one another, which represent fragments of identical size and thus identical transcripts and whose intensities differ from one another, after normalization, by at least one preselected factor, including the determined signature, in a table; in some cases, the corresponding data for fragment length (determined on the basis of the internal length standard), signal intensity and information about the amplification primers used were also included here. For general transcriptome analysis (i.e. “stock taking” of expressed genes), all determined signatures, independently of relative signal strengths, were listed in a table.

### EXAMPLE 4

### Determination of Terminal Bases with Ligation

In each case 1 μg of the purified, not fluorescently labeled amplification products of example 2 was admixed with 5 μl of 10× NEBuffer 3 and diluted with water to 49 μl. 1 μl of MboI (5 U/μl, New England Biolabs) was added and the mixture was incubated at 37° C. for 1 h; this was followed by heat-incubating at 65° C. for 20 min. The reactions were extracted, first with TE-saturated phenol, then with chloroform, and precipitated with ethanol. The pellets were taken up in 20 μl of a ligation mixture comprising 1.2 μl of 10× ligation buffer (Roche), 8 μl of 0.5 μg/μl Eco57I linker (in each case one linker selected from ECO1/2 to ECO11/12; cf. table 1; preparation of linkers by hybridizing the oligonucleotides complementary to one another, indicated in each case), and 1 μl of T4 DNA ligase (1 U/μl, Roche). Ligation was carried out at 16° C. overnight. The ligation products were amplified by mixing 2 μl of the ligation mixture with 2 μl of 10 μM amplification primer 1 (sequence-identical in each case to that strand of the Eco57I linker, whose 3′ end had been linked to the fragments cut with MboI), 2 μl of 10 μM CP31V, 5 μl of 10× Advantage 2 buffer (Clontech/BD Biosciences Europe, Heidelberg, Germany), 1 μl of 10 mM dNTPs, 37 μl of water and 1 μl of 50× Advantage 2 DNA polymerase mix (Clontech), and amplification was carried out under the following conditions: initial denaturation at 94° C. for 2 min, then 25 cycles consisting-of denaturation at 94° C. for 20 s, attaching at 65° C. for 30 s, extension at 72° C. for 2 min. After checking the amplification by means of agarose gel electrophoresis, 10 μl of the amplification products were mixed with 2.5 μl of Buffer G⁺ +SAM (Fermentas GmbH, St. Leon-Rot, Germany), 0.25 μl of 10 mg/ml BSA, 10.65 μl of water and 1.6 μl of Eco57I (5 U/μl). Incubation was carried out at 37° C. for 1 h, followed by denaturation at 65° C. for 20 min. 6.5 μl of this reaction were mixed with 1 μl of 20 mM ATP, 2 μl of 0.5 μg/μl sequencing adapter SO15NX or SO15XN (cf. table 2; preparation of linkers by hybridizing the oligonucleotides complementary to one another indicated in each case) and 0.5 μl T4 DNA ligase (1 U/μl; Roche) and incubated at 16° C. overnight. The reactions were diluted with water to 50 μl and purified by means of QiaQuick columns. Elution was carried out in 25 μl of water. In each case 2.5 μl of the purified amplication mixtures of example 2 were diluted with 9.5 μl of water and, after addition of 0.5 μl of GeneScan 500 LIZ length standard (Applied Biosystems GmbH, Weiterstadt, Germany), fractionated by capillary gel electrophoresis using the ABI 3100. For evaluation, the fluorograms of example 3 were compared with the corresponding fluorograms of example 4. Signals in fluorograms which represent the same fragment species and which had been compared with one another were identified by (1) correcting the fluorophore-specific migration behavior and (2) correcting the shortening of fragments, which increases determined base by determined base (for example by correcting the length of a fragment in which bases 3 and 4, starting from the original MboI recognition site, had been converted by Eco57I cleavage to a single-stranded protruding end arithmetically by +4 bases and correcting the length of a fragment in which bases 5 and 6, starting from the original MboI recognition site, had been converted by Eco57I cleavage to a single-stranded protruding end arithmetically by +6 bases). All signals belonging to one fragment species (i.e. a fragment appearing in example 3 and the corresponding products of example 4, which had been truncated by means of Eco57I and provided with a sequencing adapter) were assigned to one another and recorded in a table, lo and furthermore the base identity to be determined in each case was identified on the basis of the particular fluorophore. A table of this kind may have, for example, the format indicated in table 3.

The cDNA partial sequences obtained in this way (“signatures”) were used for a BLAST i5 search to identify the particular corresponding genes. It was possible, by means of the cDNA signature GATCTAGACAACCAAA retrievable from table 3, to identify the yeast gene KTR4 (ORF YBR199W) which codes for a putative alpha-1,2-mannosyl transferase. Other examples of signatures obtained from yeast can be found in FIG. 8.

TABLE 1

| | IIs | | Identified |
|---|---|---|---|
| Name | enzyme | Linker structure | bases |
| ECO1/2 | Eco57I | 5′-TCACATGCTACTGAAGCTAGTCGCGA-3′ | 1 + 2 |
| | | 3′-AGTGTACGATGACTTCGATCAGCGCTCTAG-5′ | |
| ECO3/4 | Eco57I | 5′-TCACATGCTACTCTGAAGAGTCGCGA-3′ | 3 + 4 |
| | | 3′-AGTGTACGATGAGACTTCTCAGCGCTCTAG-5′ | |
| ECO5/6 | Eco57I | 5′-TCACATGCTACTAGCTGAAGTCGCGA-3′ | 5 + 6 |
| | | 3′-AGTGTACGATGATCGACTTCAGCGCTCTAG-5′ | |
| ECO7/8 | Eco57I | 5′-TCACATGCTACTAGTCCTGAAGGCGA-3′ | 7 + 8 |
| | | 3′-AGTGTACGATGATCAGGACTTCCGCTCTAG-5′ | |
| ECO9/10 | Eco57I | 5′-TCACATGCTACTAGTCGCCTGAAGGA-3′ | 9 + 10 |
| | | 3′-AGTGTACGATGATCAGCGGACTTCCTCTAG- 5 | |
| ECO11/12 | Eco57I | 5′-TCACATGCTACTAGTCGCGACTGAAG-3′ | 11 + 12 |
| | | 3′-AGTGTACGATGATCAGCGCTGACTTCCTAG-5′ | |
| BCE1 | BceAI | 5′-TTTCACATGCACGGCTACTAGTCGCGA-3′ | 1 |
| | | 3′-CCAGTGTACGTGCCGATGATCAGCGCT-5′ | |
| BCE2 | BceAI | 5′-TTTCACATGCTACGGCACTAGTCGCGA-3′ | 2 |
| | | 3′-CCAGTGTACGATGCCGTGATCAGCGCT-5′ | |
| BCE3 | BceAI | 5′-TTTCACATGCTAACGGCCTAGTCGCGA-3′ | 3 |
| | | 3′-CCAGTGTACGATTGCCGGATCAGCGCT-5′ | |
| BCE4 | BceAI | 5′-TTTCACATGCTACACGGCTAGTCGCGA-3′ | 4 |
| | | 3′-CCAGTGTACGATGTGCCGATCAGCGCT-5′ | |
| BCE5 | BceAI | 5′-TTTCACATGCTACTACGGCAGTCGCGA-3′ | 5 |
| | | 3′-CCAGTGTACGATGATGCCGTCAGCGCT-5′ | |
| BCE6 | BceAI | 5′-TTTCACATGCTACTAACGGCGTCGCGA-3′ | 6 |
| | | 3′-CCAGTGTACGATGATTGCCGCAGCGCT-5′ | |
| BCE7 | BceAI | 5′-TTTCACATGCTACTAGACGGCTCGCGA-3′ | 7 |
| | | 3′-CCTTAGTGTACGATGATCTGCCGAGCGCT-5′ | |
| BCE8 | BceAI | 5′-TTTCACATGCTACTAGTACGGCCGCGA-3′ | 8 |
| | | 3′-CCAGTGTACGATGATCATGCCGGCGCT-5′ | |
| BCE9 | BceAI | 5′-TTTCACATGCTACTAGTCACGGCGCGA-3′ | 9 |
| | | 3′-CCAGTGTACGATGATCAGTGCCGCGCT-5′ | |
| BCE10 | BceAI | 5′-TTTCACATGCTACTAGTCGACGGCCGA-3′ | 10 |
| | | 3′-CCAGTGTACGATGATCAGCTGCCGGCT-5′ | |
| BCE11 | BceAI | 5′-TTTCACATGCTACTAGTCGCACGGCGA-3′ | 11 |
| | | 3′-CCAGTGTACGATGATCAGCGTGCCGCT-5′ | |
| BCE12 | BceAI | 5′-TTTCACATGCTACTAGTCGCGACGGCA-3′ | 12 |
| | | 3′-CCAGTGTACGATGATCAGCGCTGCCGT-5′ | |
| BCE13 | BceAI | 5′-TTTCACATGCTACTAGTCGCGAACGGC-3′ | 13 |
| | | 3′-CCAGTGTACGATGATCAGCGCTTGCCG-5′ | |

TABLE 2

| | | | Identified |
|---|---|---|---|
| Name | Linker structure | Fluorophore | base |
| NX sequencing | 5′-FAM-CTGCAGCTGGACCGANG-3′ | FAM | C |
| adapter (mixture | 3′-GACGTCGACCTGGCT-5′ | | |
| of 4 different | 5′-VIC-CTGCAGCTGGACCGANT-3′ | VIC | A |
| adapters) | 3′-GACGTCGACCTGGCT-5′ | | |
| | 5′-PET-CTGCAGCTGGACCGANA-3′ | PET | T |
| | 3′-GACGTCGACCTGGCT-5′ | | |
| | 5′-NED-CTGCAGCTGGACCGANC-3′ | NED | G |
| | 3′-GACGTCGACCTGGCT-5′ | | |
| XN sequencing | 5′-FAM-CTGCAGCTGGACCGAGN-3′ | FAM | C |
| adapter (mixture | 3′-GACGTCGACCTGGCT-5′ | | |
| of 4 different | 5′-VIC-CTGCAGCTGGACCGATN-3′ | VIC | A |
| adapters) | 3′-GACGTCGACCTGGCT-5′ | | |
| | 5′-PET-CTGCAGCTGGACCGAAN-3′ | PET | T |
| | 3′-GACGTCGACCTGGCT-5′ | | |
| | 5′-NED-CTGCAGCTGGACCGACN-3′ | NED | G |
| | 3′-GACGTCGACCTGGCT-5′ | | |

TABLE 3

| | | Corrected | | |
|---|---|---|---|---|
| | Fragment | fragment | Fluoro- | Identified |
| Experiment | length | length* | phore | base |
| “Display”** | 306 bp | 282 bp | — | GATC*** |
| Identification of base 1 | 307 bp | 280 bp | VIC | T |
| Identification of base 2 | 307 bp | 280 bp | PET | A |
| Identification of base 3 | 305 bp | 278 bp | FAM | G |
| Identification of base 4 | 305 bp | 278 bp | PET | A |
| Identification of base 5 | 303 bp | 276 bp | NED | C |
| Identification of base 6 | 303 bp | 276 bp | PET | A |
| Identification of base 7 | 301 bp | 274 bp | PET | A |
| Identification of base 8 | 301 bp | 274 bp | NED | C |
| Identification of base 9 | 299 bp | 272 bp | NED | C |
| Identification of base 10 | 299 bp | 272 bp | PET | A |
| Identification of base 11 | 297 bp | 270 bp | PET | A |
| Identification of base 12 | 297 bp | 270 bp | PET | A |
| *Double-stranded portion of the fragment after arithmetical removal of the linker, corrected for the contribution of the fluorophore to electrophoretic fragment mobility. The numbers in this example refer to the use of Eco57I which generates two-base protruding ends for identification of in each case two adjacent bases (“doublets”) and of sequencing adapters which identify alternatively the first or the second base of such a protruding end. To identify a plurality of successive | | | | |
| # doublets, the recognition sites for Eco57I, located in the Eco57I linkers, are in each case staggered by two bases. | | | | |

**Reaction according to example 3
***Resulting from the known recognition site of MboI (cf. example 1)

### EXAMPLE 5

### Determination of Terminal Bases via Fill-In Reaction

In each case 1 μg of the purified, not fluorescently labeled amplification products of example 2 was admixed with 5 μl of 10× NEBuffer 3 and diluted with water to 49 μl. 1 μl of MboI (5 U/μl, New England Biolabs) was added and the mixture was incubated at 37° C. for 1 h; this was followed by heat-incubating at 65° C. for 20 min. The reactions were extracted, first with TE-saturated phenol, then with chloroform, and precipitated with ethanol. The pellets were taken up in 50 μl of mung bean nuclease buffer (New England Biolabs). Addition of 1 μl of mung bean nuclease (1 U/μl, New England Biolabs) was followed by incubation at 30° C. for 30 min. 1 μl of 0.5 M EDTA was added, followed by extraction with phenol, then with chloroform, and precipitation with ethanol. The precipitate was dissolved in a ligation mixture of 7.5 μl of 2× ligation buffer (New England Biolabs), 6.5 μl of 0.5 μg/μl BceAI linker (in each case one linker, selected from BCE1 to BCE13; cf. table 1; preparation of linkers by hybridization of the oligonucleotides complementary to one another indicated in each case), and 2 μl of Quick T4 DNA ligase (New England Biolabs), followed by ligation at room temperature for 1 h. The ligation products were amplified by mixing 2 μl of the ligation with 2 μl of 10 μM amplification primer 2 (sequence-identical to in each case that strand of the BceAI linker, whose 3′ end had been linked to the fragments cut with MboI), 2 μl of 10 μM CP31, 5 μl of 10× Advantage 2 buffer, 1 μl of 10 mM dNTPs, 37 μl of water and I pi of 50× Advantage 2 DNA polymerase mix, and the amplification was carried out under the following conditions: initial denaturation at 94° C. for 2 min, then 25 cycles consisting of denaturation at 94° C. for 20 s, attaching at 65° C. for 30 s, extension at 72° C. for 2 min. After check the amplification by means of agarose gel electrophoresis, 10 μl of the amplification products were mixed with 3 μl of NEBuffer BceAI (New England Biolabs), 0.3 μl of 10 mg/ml BSA, 13.7 μl of water and 3 μl of BceAI (1 U/μl). An incubation was carried out at 37° C. for 4 h, followed by denaturation at 65° C. for 20 min. 9 μl of this reaction were mixed with 1 μl of ddNTP mix (in each case 10 mM FAM-ddATP, JOE-ddTTP, ROX-ddATP and TAMRA-ddCTP, PerkinElmer Life Sciences Inc., Boston) and 0.5 μl of Klenow polymerase (5 U/μl, New England Biolabs) and incubated at 37° C. for 5 min. After stopping the reaction with EDTA and heat-denaturation at 75° C. for 20 min, the solution was diluted with water to 50 μl and purified by means of QiaQuick columns. Fractionation, evaluation and analysis of the data were carried out analogously to example 4.

1-26. (canceled)

27. A method of analyzing nucleic acid fragment mixtures, comprising the steps
(a) providing at least one mixture of those nucleic acid fragments which have at least one recognition site for a restriction endonuclease cutting outside its recognition site,
(b) incubating at least a subset of said mixture of nucleic acid fragments of step (a) with at least one restriction endonuclease whose cleavage site is located outside its recognition site, and
(c) identifying one or more nucleotides of the cut nucleic acid fragments of (b) and, where appropriate, identifying further fragment-specific properties of said cut nucleic acid fragments of (b), said identification(s) being carried out simultaneously for a plurality of or for all nucleic acid fragments.

28. The method as claimed in claim 27, wherein the identification in step (c) additionally comprises fractionating the cut nucleic acid fragments according to fragment-specific properties.

29. The method as claimed in claim 28, wherein the cut nucleic acid fragments are fractionated according to fragment-specific properties by means of gel electrophoresis.

30. The method as claimed in claim 29, wherein the fractionation is carried out by means of capillary electrophoresis.

31. The method as claimed in claim 27, wherein the method step (c) comprises the following individual steps (ca) to (cd):
(ca) identifying in each case a first nucleotide of the cut nucleic acid fragments of (b), said identification being carried out simultaneously for a plurality of or all nucleic acid fragments,
(cb) identifying, where appropriate, in each case a further nucleotide of said cut nucleic acid fragments of (b), said identification being carried out simultaneously for a plurality of or all nucleic acid fragments,
(cc) repeating, where appropriate, step (cb), until the desired number of nucleotides have been identified, and
(cd) combining the sequence information obtained in steps (ca) to (cc) for a selected group or for all nucleic acid fragments to fragment-specific signatures, with a signature being able to contain, in addition to said sequence information, also further information about the particular fragment,
with the nucelotide identification in steps (ca) to (cc), where appropriate, additionally also comprising fractionating the nucleic acid fragments of the mixture.

32. The method as claimed in claim 27, wherein a subset of the mixture of nucleic acid fragments provided in step (a), which subset is different from the subset to be incubated in step (b), is subjected to the following method steps (aa) to (ad):
(aa) fractionating the mixture of nucleic acid fragments according to at least one fragment-specific property,
(ab) detecting, where appropriate, the relative frequency of some or all fragments in the mixture fractionated in (aa),
(ac) comparing, where appropriate, the information obtained in (aa) and (ab) or the information obtained in (aa) or (ab) about the composition of various mixtures of nucleic acid fragments of step (a), and
(ad) registering, where appropriate, nucleic acid fragments detected in (ab) which occur with different relative frequencies in various mixtures of nucleic acid fragments,
while another subset selected from the group consisting of (I) to (II) is treated according to steps (b) and (c), with
(I) being a further subset of the mixture of nucleic acid fragments provided in step (a),
(II) being a subset of the mixture of nucleic acid fragments provided in step (a) which has previously been fractionated according to at least one fragment-specific property, and
(III) being a mixture of nucleic acid fragments which is at least partially identical to (I) or (II).

33. The method as claimed in claim 27, wherein an additional method step comprises isolating at least one fragment of interest either:
(a) from the mixture of nucleic acid fragments of (a); or
(b) from a mixture of nucleic acid fragments of (a) which have previously been fractionated according to a fragment-specific property.

34. The method as claimed in claim 31, wherein an additional method step comprises isolating at least one fragment of interest either:
(a) from the mixture of nucleic acid fragments of (a); or
(b) from a mixture of nucleic acid fragments of (a) which have previously been fractionated according to a fragment-specific property.

35. The method as claimed in claim 34, wherein the additional method step comprises:
(a) isolating fragments by preparing fragment-specific oligonucleotide primers,
(b) using the signatures determined in step (cd), and
(c) then using said oligonucleotide primers for specific amplification of said fragments from the mixture of nucleic acid fragments by means of PCR.

36. The method as claimed in claim 31, wherein the signatures, obtained in step (cd), of individual nucleic acid fragments of the fragment mixture are used in a database search for identifying these fragments.

37. The method as claimed in claim 27, wherein the mixture of nucleic acid fragments of (a) is a mixture of cDNA fragments or a mixture of fragments of genomic DNA.

38. The method as claimed in claim 27, wherein the mixture of nucleic acid fragments of (a) comprises restriction fragments produced by incubating a nucleic acid mixture with at least one restriction enzyme.

39. The method as claimed in claim 38, wherein as the mixture of nucleic acid fragments of (a) at least one further subset is provided prepared by the following steps:
(i) flanking of the restriction fragments of the mixture on either side by identical or different adapters;
(ii) hybridizing the fragments of step (i) with in each case different primers all of which have regions complementary to the adapters of step (i) and whose 3′ end has in each case one or more nucleotides which protrude beyond the region complementary to the adapter and which are complementary to a subset of the fragments of the nucleic acid mixture of (a); and
(iii) sequence-specific extension of the primers of (ii) and, where appropriate, subsequent PCR amplification of the nucleic acid fragments of the fragment mixture, which had been extended sequence-specifically.

40. The method as claimed claim 27, which comprises providing the mixture of nucleic acid fragments of step (a) by ligating the particular nucleic acid fragments of the fragment mixture to be analyzed with one or more linkers which have in at least one specific position at least one recognition site for a restriction endonuclease whose cleavage site is outside its recognition site.

41. The method as claimed in claim 40, wherein the particular nucleic acid fragments of the fragment mixture to be analyzed are ligated with in each case a plurality of different linkers which differ from one another in the position of the recognition site for a restriction endonuclease whose cleavage site is outside its recognition site.

42. The method as claimed in claim 27, wherein identification of one or more nucleotides of the cut nucleic acid fragments of (b), which identification takes place simultaneously for a plurality of or all nucleic acid fragments, is carried out via filling protruding ends with termination nucleotides carrying labeling groups.

43. The method as claimed in claim 27, wherein identification of one or more nucleotides of the cut nucleic acid fragments of (b), which identification takes place simultaneously for a plurality of or all nucleic acid fragments in step (c), is carried out via the following steps (cm) to (cp):
(cm) hybridizing in each case one strand of the nucleic acid fragments of (b) with selective oligonucleotide primers whose nucleotide or nucleotides located at the 3′ end can hybridize with the nucleotide(s) to be sequenced of the particular strand;
(cn) extending said selective oligonucleotide primers; and
(cp) identifying those selective oligonucleotide primers which have been extended in step (cn).

44. The method as claimed in claim 27, wherein one or more nucleotides of the cut nucleic acid fragments of (b) are identified in parallel via the sequence-specific attachment of adapters with protruding ends of suitable length and type, which adapters differ from one another with respect to their protruding ends.

45. The method as claimed in claim 44, wherein the protruding ends of the adapters used comprise a degenerate portion and a portion having a defined sequence.

46. The method as claimed in claim 44, wherein the adapters used whose protruding ends comprise different portions having a defined sequence are labeled differently.

47. The method as claimed in claim 45, wherein the adapters used whose protruding ends comprise different portions having a defined sequence are labeled differently.

48. The method as claimed in of claim 27, wherein it is used for cataloguing nucleic acid signatures.

49. The method as claimed in claim 27, wherein it is used for generating EST libraries.

50. The method as claimed in claim 27, wherein it is used for identifying genes which are differentially expressed between at least two biological samples.

51. The method as claimed in claim 50, wherein:
(A) method step (a) comprises the following substeps (a1) to (e1) which are as follows:
(a1) providing at least one mixture of nucleic acid fragments, in particular at least one mixture of cDNA fragments,
(b1) fractionating the mixture of nucleic acid fragments of a1) according to at least one fragment-specific property,
(c1) detecting, where appropriate, the relative frequency of some or all fragments in the fractionated mixture of b1),
(d1) comparing, where appropriate, the information obtained in (b1) and (c1) or the information obtained in (b1) or (c1) about the composition of various mixtures of nucleic acid fragments of (a1), and
(e1) registering, where appropriate, nucleic acid fragments detected in (d1) which appear in various mixtures of nucleic acid fragments with different relative frequencies;
(B) method step (b) is replaced by method step (f1) which is as follows:
(f1) incubating a mixture of nucleic acid fragments selected from
the group I: a subset of the mixture of (a1),
the group II: the mixture of cDNA fragments fractionated in (b1) or a part thereof,
the group III: a mixture of nucleic acid fragments which is at least partially identical to the mixture of (a1) or to the fractionated mixture of (b1), but which additionally has at least one recognition site for a restriction endonuclease cutting outside its recognition site, with at least one restriction endonuclease cutting outside its recognition site;
(C) method step (c) comprises the following substeps (g1) to (k1) which are as follows:
(g1) identifying a first nucleotide of the cut nucleic acid fragments of (f1), said identification being carried out simultaneously for a plurality of or all nucleic acid fragments,
(h1) identifying, where appropriate, a further nucleotide of the cut nucleic acid fragments of (f1), said identification being carried out simultaneously for a plurality of or all nucleic acid fragments,
(i1) repeating, where appropriate, step (h1), until the desired number of nucleotides have been identified,
(j1) repeating, where appropriate, once or several times steps (f1) to (i1), with the position and sequence or with the position or sequence of the recognition site being varied in each case in such a way that repeating steps (f1) to (i1) allows in each case nucleotides to be identified which have not been identified previously, and
(k1) combining the sequence information, obtained in steps (g1) to (j1), for all nucleic acid fragments or for a selected group of said nucleic acid fragments to give fragment-specific signatures, with a signature, where appropriate, containing, in addition to said sequence information, still further information about the particular fragment; and
(D) where appropriate, additionally at least one of the optional steps (l1) and (m1) is carried out, with (l1) and (m1) being as follows:
(l1) obtaining fragments of interest from the mixture of nucleic acid fragments of (a1) or (b1), said fragments of interest preferably being the fragments registered in (e1), and
(m1) identifying the genes corresponding to the nucleic acid fragments of interest, from which said nucleic acid fragments are derived, by means of screening electronic databases, said fragments of interest preferably being the fragments registered in (e1).

52. The method as claimed in claim 50, wherein:
(A) method step (a) is replaced by the method step (a2) which is as follows:
(a2) providing at least one mixture of nucleic acid fragments, which has a linker and, within the sequence of said linker, at least one recognition site for at least one restriction endonuclease cutting outside its recognition site,
(B) method step (b) is replaced by the method step (b2) which is as follows:
(b2) incubating the mixture of nucleic acid fragments of (a2) with the at least one restriction endonuclease of step (a2),
(C) method step (c) comprises the substeps (c2) to (i2) which are as follows:
(c2) identifying a first nucleotide of the cut nucleic acid fragments of (b2), said identification being carried out simultaneously for a plurality of or all nucleic acid fragments of the mixture and with fractionation of the mixture of cut nucleic acid fragments according to at least one fragment-specific property,
(d2) identifying, where appropriate, a further nucleotide of the cut nucleic acid fragments of (b2) according to step (c2),
(e2) repeating, where appropriate, step (d2), until the desired number of nucleotides has been identified,
(f2) repeating, where appropriate, once or several times steps (a2) to (e2), with the position and sequence or with the position or sequence of the recognition site having been modified in each case in such a way that their repetition allows in each case nucleotides to be identified which have not been identified previously,
(g2) combining the sequence information, obtained in steps (c2) to (f2), for all nucleic acid fragments or for a selected group of said nucleic acid fragments to give fragment-specific signatures, it being possible for a signature to contain, in addition to said sequence information, still further information about the particular fragment,
(h2) assigning the fragment-specific information obtained from the fractionation according to a fragment-specific property in (c2) to the signatures obtained for the nucleic acid fragments in (g2), said fragment-specific information comprising, in the case of an electrophoretic fractionation of the fragments, the relative or absolute mobility of said fragments and the apparent or actual fragment length or the relative or absolute mobility of said fragments or the apparent or actual fragment length determined on the basis of a length standard and it being possible for said assigning to be done in table form and in a computer-readable form or to be done in table form or a computer-readable form, and
(i2) identifying, where appropriate, the genes corresponding to the nucleic acid fragments, from which said nucleic acid fragments are derived, by means of screening electronic databases for the signatures of (g2);
and additionally carrying out at least one of steps (j2) to (p2), with (j2) and (p2) being as follows:
(j2) providing, where appropriate, at least one further mixture of nucleic acid fragments, obtained in an analogous way to the mixture of nucleic acid fragments of (a2), it being possible here to dispense with the adding of linkers having at least one recognition site for a restriction endonuclease cutting outside its recognition site,
(k2) fractionating the mixture of nucleic acid fragments of (j2) according to a fragment-specific property,
(l2) assigning the fragment-specific information obtained from the fractionation according to a fragment-specific property in (k2) to the individual fractionated fragments,
(m2) comparing, where appropriate, the relative or absolute frequencies of at least part of the fragments fractionated in (k2) to the relative or absolute frequencies of in each case homologous fragments derived from other nucleic acid fragment mixtures,
(n2) registering, where appropriate, those fragments whose relative or absolute frequency differs from the relative or absolute frequency of their homologous fragments derived from other nucleic acid fragment mixtures,
(o2) assigning, where appropriate, the fragments registered in (n2) to those genes or transcripts from which said registered fragments are derived, and
p2) obtaining, where appropriate, the fragments registered in (n2) from the mixture of nucleic acid fragments of (a2) or (i2) and (j2) or from the mixture of nucleic acid fragments of (a2) or (i2) or (j2),
it also being possible for steps (i2) to (n2) to be carried out before steps (a2) to (h2).

53. A method of analyzing nucleic acid fragment mixtures, comprising the steps
(a) providing a mixture of nucleic acid fragments which have at least one recognition site for a restriction endonuclease cutting outside its recognition site,
(b) incubating at least a subset of the mixture of nucleic acid fragments of step (a) with at least one restriction endonuclease whose cleavage site is located outside its recognition site and which generates protruding ends of known position and length, but unknown sequence,
(c) identifying in each case one or more nucleotides of said protruding ends of the cut nucleic acid fragments of (b) and, where appropriate, identifying further fragment-specific properties of said cut nucleic acid fragments of (b), said identification(s) being carried out simultaneously for a plurality of or for all nucleic acid fragments.


SEQUENCE LISTING (52 sequences)

SEQ ID NO: 1 - DNA, 32 bp, artificial sequence
  primer CP31V (example 1)
acctacgtgcagattttttttttttttttttv

SEQ ID NO: 2 - DNA, 20 bp, artificial sequence
  Oligonucleotide ML20 (example 1)
tcacatgctaagtctcgcga

SEQ ID NO: 3 - DNA, 25 bp, artificial sequence
  oligonucleotide LM25 (example 1)
gatctcgcgagacttagcatgtgac

SEQ ID NO: 4 - DNA, 31 bp, artificial sequence
  primer  CP31X1X2 (example 2), at the 3′ end followed by nucleotide X1 which can be G, A, C and by X2 followed by nucleotide G, A, T
acctacgtgcagatttttttttttttttttt

SEQ ID NO: 5 - DNA, 31 bp, artificial sequence; identical to SEQ ID NO: 4
  primer CP31VNX3X4, at the 3′ end followed by V=mixture of G, A, C, followed by N=mixture of G, A, T, followed by X3 and X4 = G, A, T, C (example 2)

SEQ ID NO: 6 - DNA, 26 bp, Artificial sequence
  sense-strand for adaptor ECO1/2
tcacatgctactgaagctagtcgcga

SEQ ID NO: 7 - DNA, 30 bp, artificial sequence
  as-strand for adaptor ECO1/2
gatctcgcgactagcttcagtagcatgtga

SEQ ID NO: 8 - DNA, 26 bp, artificial sequence
  sense-strand for adaptor ECO3/4
tcacatgctactctgaagagtcgcga

SEQ ID NO: 9 - DNA, 30 bp, artificial sequence
  as-strand for adaptor ECO3/4
gatctcgcgactcttcagagtagcatgtga

SEQ ID NO: 10 - DNA, 26 bp, artificial sequence
  sense-strand for adaptor ECO5/6
tcacatgctactagctgaagtcgcga

SEQ ID NO: 11 - DNA, 30 bp, artificial sequence
  as-strand for adaptor ECO5/6
gatctcgcgacttcagctagtagcatgtga

SEQ ID NO: 12 - DNA, 26 bp, artificial sequence
  sense-strand for adaptor ECO7/8
tcacatgctactagtcctgaaggcga

SEQ ID NO: 13 - DNA, 30 bp, artificial sequence
  as-strand for adaptor ECO7/8
gatctcgccttcaggactagtagcatgtga

SEQ ID NO: 14 - DNA, 26 bp, artificial sequence
  sense-strand for adaptor ECO9/10
tcacatgctactagtcgcctgaagga

SEQ ID NO: 15 - DNA, 30 bp, artificial sequence
  as-strand for adaptor ECO9/10
gatctccttcaggcgactagtagcatgtga

SEQ ID NO: 16 - DNA, 26 bp, artificial sequence
  sense-strand for adaptor ECO11/12
tcacatgctactagtcgcgactgaag

SEQ ID NO: 17 - DNA, 30 bp, artificial sequence
  as-strand for adaptor ECO11/12
gatccttcagtcgcgactagtagcatgtga

SEQ ID NO: 18 - DNA, 27 bp, artificial sequence
  sense-strand for adaptor BCE1
tttcacatgcacggctactagtcgcga

SEQ ID NO: 19 - DNA, 27 bp, artificial sequence
  as-strand for adaptor BCE1
tcgcgactagtagccgtgcatgtgacc

SEQ ID NO: 20 - DNA, 27 bp, artificial sequence
  sense-strand for adaptor BCE2
tttcacatgctacggcactagtcgcga

SEQ ID NO: 21 - DNA, 27 bp, artificial sequence
  as-strand for adaptor BCE2
tcgcgactagtgccgtagcatgtgacc

SEQ ID NO: 22 - DNA, 27 bp, artificial sequence
  sense-strand for adaptor BCE3
tttcacatgctaacggcctagtcgcga

SEQ ID NO: 23 - DNA, 27 bp, artificial sequence
  as-strand for adaptor BCE3
tcgcgactaggccgttagcatgtgacc

SEQ ID NO: 24 - DNA, 27 bp, artificial sequence
  sense-strand for adaptor BCE4
tttcacatgctacacggctagtcgcga

SEQ ID NO: 25 - DNA, 27 bp, artificial sequence
  as-strand for adaptor BCE4
tcgcgactagccgtgtagcatgtgacc

SEQ ID NO: 26 - DNA, 27 bp, artificial sequence
  sense-strand for adaptor BCE5
tttcacatgctactacggcagtcgcga

SEQ ID NO: 27 - DNA, 27 bp, artificial sequence
  as-strand for adaptor BCE5
tcgcgactgccgtagtagcatgtgacc

SEQ ID NO: 28 - DNA, 27 bp, artificial sequence
  sense-strand for adaptor BCE6
tttcacatgctactaacggcgtcgcga

SEQ ID NO: 29 - DNA, 27 bp, artificial sequence
  as-strand for adaptor BCE6
tcgcgacgccgttagtagcatgtgacc

SEQ ID NO: 30 - DNA, 27 bp, artificial sequence
  sense-strand for adaptor BCE7
tttcacatgctactagacggctcgcga

SEQ ID NO: 31 - DNA, 29 bp, artificial sequence
  as-strand for adaptor BCE7
tcgcgagccgtctagtagcatgtgattcc

SEQ ID NO: 32 - DNA, 27 bp, artificial sequence
  sense-strand for adaptor BCE8
tttcacatgctactagtacggccgcga

SEQ ID NO: 33 - DNA, 27 bp, artificial sequence
  as-strand for adaptor BCE8
tcgcggccgtactagtagcatgtgacc

SEQ ID NO: 34 - DNA, 27 bp, artificial sequence
  sense-strand for adaptor BCE9
tttcacatgctactagtcacggcgcga

SEQ ID NO: 35 - DNA, 27 bp, artificial sequence
  as-strand for adaptor BCE9
tcgcgccgtgactagtagcatgtgacc

SEQ ID NO: 36 - DNA, 27 bp, artificial sequence
  sense-strand for adaptor BCE10
tttcacatgctactagtcgacggccga

SEQ ID NO: 37 - DNA, 27 bp, artificial sequence
  as-strand for adaptor BCE10
tcggccgtcgactagtagcatgtgacc

SEQ ID NO: 38 - DNA, 27 bp, artificial sequence
  sense-strand for adaptor BCE11
tttcacatgctactagtcgcacggcga

SEQ ID NO: 39 - DNA, 27 bp, artificial sequence
  as-strand for adaptor BCE11
tcgccgtgcgactagtagcatgtgacc

SEQ ID NO: 40 - DNA, 27 bp, artificial sequence
  sense-strand for adaptor BCE12
tttcacatgctactagtcgcgacggca

SEQ ID NO: 41 - DNA, 27 bp, artificial sequence
  as-strand for adaptor BCE12
tgccgtcgcgactagtagcatgtgacc

SEQ ID NO: 42 - DNA, 27 bp, artificial sequence
  sense-strand for adaptor BCE13
tttcacatgctactagtcgcgaacggc

SEQ ID NO: 43 - DNA, 27 bp, artificial sequence
  as-strand for adaptor BCE13
gccgttcgcgactagtagcatgtgacc

SEQ ID NO: 44 - DNA, 17 bp, artificial sequence
  primer of table 2, linked to the dye FAM at its 5′ end
ctgcagctggaccgang

SEQ ID NO: 45 - DNA, 17 bp, artificial sequence
  primer of table 2, linked to the dye VIC at its 5′ end
ctgcagctggaccgant

SEQ ID NO: 46 - DNA, 17 bp, artificial sequence
  primer of table 2; linked to the dye PET at its 5′ end
ctgcagctggaccgana

SEQ ID NO: 47 - DNA, 17 bp, artificial sequence
  primer of table 2; linked to the dye NED at its 5′ end
ctgcagctggaccganc

SEQ ID NO: 48 - DNA, 17 bp, artificial sequence
  primer of table 2; linked to the dye FAM at its 5′ end
ctgcagctggaccgagn

SEQ ID NO: 49 - DNA, 17 bp, artificial sequence
  primer of table 2; linked to the dye VIC at its 5′ end
ctgcagctggaccgatn

SEQ ID NO: 50 - DNA, 17 bp, artificial sequence
  primer of table 2; linked to the dye PET at its 5′ end
ctgcagctggaccgaan

SEQ ID NO: 51 - DNA, 17 bp, artificial sequence
  primer of table 2; linked to the dye NED at its 5′ end
ctgcagctggaccgacn

SEQ ID NO: 52 - DNA, 15 bp, artificial sequence
  primer of table 2
tcggtccagctgcag